SEQUENCE LISTING (2 sequences)

SEQ ID NO: 1 - protein, 1185 aa, Artificial Sequence
  Synthetic
  MISC_FEATURE: Cry14Ab1 amino acid sequence
MDCNLQSQQNIPYNVLAIPVSNVNSLTDTVGDLKKAWEEFQKTGSFSLTALQQGFSASQGGTFNYLTLLQSGISLAGSFVPGGTFVAPIINMVIGWLWPHKNKNADTENLINLIDSEIQKQLNKALLDADRNEWSSYLESIFDSSNNLNGAIVDAQWSGTVNTTNRTLRNPTESDYTNVVTNFIAADGDIANNENHIMNGNFDVAAAPYFVIGATARFAAMQSYIKFCNAWIDKVGLSDAQLTTQKANLDRTKQNMRNAILNYTQQVMKVFKDSKNMPTIGTNKFSVDTYNVYIKGMTLNVLDIVAIWPSLYPDDYTSQTALEQTRVTFSNMVGQEEGTDGSLRIYNTFDSFSYQHSPIPNNNVNLISYYNDELQNLELGVYTPPKKGSGYSYPYGFVLNYANSKYKYGDSNDPESLGGLSTLSAPIQQVNAATQNSKYLDGEILNGIGASLPGYCTTGCSPTEPPFSCTSTANGYKASCNPSDTNQKINALYPFTQANVKGNTGKLGVLASLVSYDLNPKNVFGELDSDTNNVILKGIPAEKGYFPNNARPTVVKEWINGASAVPLDSGNTLFMTATNLTATQYRIRIRYANPNSNTQIGVRITQNGSLISSSNLTLYSTTDMNNTLPLNVYVIGENGNYTLQDLYNTTNVLSTGDITLQITGGDQKIFIDRIEFVPTMPVPGNTNNNNGNNNGNNNPPHHVCAIAGTQQSCSGPPKFEQVSDLEKITTQVYMLFKSSPYEELALEVSSYQISQVALKVMALSDELFCEEKNVLRKLVNKAKQLLEASNLLVGGNFETTQNWVLGTNAYINYDSFLFNGNYLSLQPASGFFTSYAYQKIDESTLKPYTRYKVSGFIGQSNQVELIISRYGKEIDKILNVPYAGPLPITADASITCCAPEIGQCDGEQSDSHFFNYSIDVGALHPELNPGIEIGLKIVQSNGYITISNLEIIEERPLTEMEIQAVNRKNQKWEREKLLECASISELLQPIINQIDSLFKDGNWYNDILPHVTYQDLKNIIIPELPKLKHWFIENLPGEYHEIEQKMKEALKYAFTQLDEKNLIHNGHFTTNLIDWQVEGDAQMKVLENDALALQLFNWDASASQSINILEFDEDKAYKLRVYAQGSGTIQFGNCEDEAIQFNTNSFIYQEKIVYFDTPSVNLHIQSEGSEFIVSSIDLIELSDDQ

SEQ ID NO: 2 - DNA, 3558 bp, Artificial Sequence
  Synthetic
  misc_feature: Cry14Ab1 nucleotide sequence
atggattgcaaccttcagtcccagcagaacattccatacaacgtgctcgctattccagtttctaacgtgaactcccttactgataccgtgggtgatcttaagaaggcttgggaagagttccaaaagaccggatctttctctcttactgctctccaacagggattctctgcttctcaaggtggaaccttcaactaccttacccttctccagtctggaatttctcttgctggatccttcgttccaggtggaactttcgtggctccaatcatcaacatggtgattggatggctttggccacacaagaacaagaacgctgataccgagaacctcattaacctcatcgattccgagattcagaagcagcttaacaaggctcttctcgatgctgataggaacgagtggtcctcttaccttgagtccatcttcgattcctccaacaacctcaacggtgctattgtggatgctcagtggagtggaactgttaacactaccaacaggacccttagaaacccaaccgagtccgattacaccaacgttgtgaccaacttcattgctgctgatggcgatattgccaacaacgagaaccacatcatgaacggaaacttcgatgttgctgctgctccatacttcgttattggagctaccgctagattcgctgctatgcaatcctacatcaagttctgcaacgcttggattgacaaagtgggactttccgatgctcaacttactacccagaaggctaaccttgataggaccaagcagaacatgaggaacgctatccttaactacacccagcaggttatgaaggtgttcaaggactccaagaacatgccaaccattggcaccaacaagttctctgtggacacctacaacgtgtacatcaagggcatgaccttgaacgtgctcgatattgtggctatttggccatccctttacccagatgattacacctctcagactgctcttgagcaaactagggtgaccttctctaacatggtgggtcaagaagaaggtactgacggatctctcaggatctacaacaccttcgactcattctcttaccagcactccccaatcccaaacaacaacgtgaacctcatctcctactacaacgacgagcttcagaaccttgagcttggagtttacaccccaccaaagaagggatctggatactcttacccatacggcttcgtgcttaactacgccaactccaagtacaagtacggcgattctaacgatccagagtctcttggaggactttctaccctttccgctccaattcaacaggttaacgctgctacccagaactctaagtacctcgatggcgagattcttaacggaattggagcttcccttccaggatattgcactactggatgctctccaactgaaccaccattctcttgcacttctaccgctaacggatacaaggcttcttgcaacccatctgacaccaaccagaagatcaacgctctttacccattcactcaggctaacgtgaagggaaacaccggaaagcttggagttcttgcttctctcgtgtcctacgatctcaacccaaagaacgtgttcggagagcttgattccgataccaacaacgtgattctcaagggaattccagctgagaagggctatttcccaaacaacgctaggccaaccgttgtgaaagagtggattaacggcgcttctgctgttccacttgattctggcaacacccttttcatgaccgctactaaccttactgctacccagtacaggattaggatcagatacgccaacccaaactccaacacccaaatcggagttaggattacccagaacggatcccttatttcttcttccaacctcaccctttactctaccaccgacatgaacaacacccttccacttaacgtgtacgtgattggagagaacggaaactacacccttcaggacctttacaacaccaccaacgtgctttctaccggtgatattaccctccaaatcaccggtggagatcagaagattttcatcgacaggatcgagttcgttccaactatgccagttccaggcaacactaacaacaacaacggaaacaacaatggcaacaataacccaccacatcatgtgtgtgctattgctggaactcagcagtcttgttctggaccaccaaagttcgagcaagtgtccgatcttgagaagattaccacccaggtgtacatgcttttcaagtcctccccatacgaagaacttgctcttgaggtgtcctcttaccagatttcccaagtggctcttaaggtgatggctctctccgatgaacttttctgcgaagagaagaacgtgcttaggaagcttgtgaacaaggccaagcaacttcttgaggcttccaaccttcttgttggaggcaacttcgagactactcagaactgggtgttgggaactaacgcctacatcaactacgattccttcctcttcaacggtaactacctttctcttcagccagcttctggattcttcacctcctacgcctaccaaaagattgatgagtccacccttaagccatacaccaggtacaaggtgtcaggattcattggacagtctaaccaggtggagcttatcatttccagatacggcaaagagatcgacaagatcctcaacgttccatatgctggaccacttccaattaccgctgatgcttccattacttgctgcgctccagaaattggacaatgcgacggcgaacagtctgattctcacttcttcaactactccatcgatgtgggtgctcttcatccagaactcaacccaggaattgagatcggactcaagatcgttcagtccaacggttacatcaccatttccaacctcgagatcattgaggaaaggccacttaccgagatggaaatccaggctgtgaataggaagaaccagaagtgggagagggaaaagcttcttgagtgcgcttctatttctgagcttctccagcctatcatcaaccagattgactccctcttcaaggatggaaactggtacaacgatatccttccacatgtgacctaccaggacctcaagaacattatcatcccagagcttccaaagcttaagcactggttcattgagaacttgcctggtgagtaccatgagatcgagcagaagatgaaggaagctctcaagtacgctttcacccagcttgatgagaagaacctcattcacaacggacatttcaccaccaacctcattgattggcaagttgagggtgatgctcagatgaaggtgttggagaacgatgctcttgctcttcagctcttcaactgggatgcttctgcttcccagtccattaacatcctcgagttcgatgaggataaggcttacaagcttagggtttacgctcaaggatctggaactatccagttcggaaactgcgaagatgaggccattcagttcaacaccaacagcttcatctaccaagagaagatcgtgtacttcgataccccatctgtgaaccttcacattcagtctgagggatccgagttcattgtgtcctccatcgatctcattgagctttccgacgaccagtga

(19) United States
(12) Patent Application Publication
(10) Pub. No.: US 2024/0206477 A1
(43) Pub. Date: Jun. 27, 2024

(54) Nematode Suppression

(71) Applicant: BASF Agricultural Solutions Seed US LLC, Florham Park, NJ (US)

(72) Inventors: Michael McCarville, Morrisville, NC (US); Julia Daum, Morrisville, NC (US); Rodrigo Moreira Saraiva, Morrisville, NC (US)

(73) Assignee: BASF Agricultural Solutions Seed US LLC, Florham Park, NJ (US)

(21) Appl. No.: 18/554,095

(22) PCT Filed: Apr. 8, 2022
(86) PCT No.: PCT/US2022/024113

§ 371 Date: Oct. 5, 2023

Related U.S. Application Data
(60) Provisional application No. 63/174,191, filed on Apr. 13, 2021.

(51) Int. Cl. A01N 63/50; A01C 14/00; A01N 63/23; A01P 5/00

(52) U.S. Cl. CPC A01N 63/50; A01C 14/00; A01N 63/23; A01P 5/00

(57) ABSTRACT

The present disclosure is directed to nematode suppression methods. Methods of increasing yield of a nematode-susceptible plant are also provided.

### CROSS-REFERENCE TO RELATED APPLICATIONS

This application claims the priority benefit under 35 U.S.C. § 1.19(e) of U.S. Provisional Patent Application No. 63/174,191, filed Apr. 13, 2021, which is incorporated herein by reference in its entirety.

### FIELD

The present disclosure is directed to nematode suppression methods. Methods of increasing yield of a nematode-susceptible plant are also provided.

### INCORPORATION BY REFERENCE OF MATERIAL SUBMITTED ELECTRONICALLY

This application contains, as a separate part of the disclosure, a Sequence Listing in computer-readable form which is incorporated by reference in its entirety and identified as follows: Filename: 202662_Seglisting.txt; Size 15,423 bytes; Created: Mar. 21, 2022.

### BACKGROUND

Nematodes are active, flexible, elongate, organisms that live on moist surfaces or in liquid environments, including films of water within soil and moist tissues within other organisms. Many species of nematodes have evolved to be very successful parasites of plants and animals and are responsible for significant economic losses in agriculture and livestock and for morbidity and mortality in humans (Whitehead (1998) Plant Nematode Control. CAB International, New York).

It is estimated that parasitic nematodes cost the horticulture and agriculture industries in excess of $78 billion worldwide a year, based on an estimated average 12% annual loss spread across all major crops. For example, it is estimated that nematodes cause soybean losses of approximately $3.2 billion annually worldwide (Barker et al. (1994) Plant and Soil Nematodes: Societal Impact and Focus for the Future. The Committee on National Needs and Priorities in Nematology. Cooperative State Research Service, US Department of Agriculture and Society of Nematologists). Plant-parasitic nematodes are pests of all major food commodities worldwide, including corn, barley, sorghum, oats, rye, rice, potatoes, cassava, sweet potatoes, wheat, soybeans, rapeseed, and sunflower (Nicol et al. (2011), in addition to being important pests of fruit and vegetable crops, fiber crops (e.g. cotton), ornamentals, and turf grass (Current Nematode Threats to World Agriculture. In: J. Jones et al. (eds.) Genomics and Molecular Genetics of Plant-Nematode Interactions, Springer Science+Business Media B. V. 2011).

Nematodes are known to affect the yield, growth, and health of crops and plants. The physiological changes in the host plant's roots caused by larvae and/or adult nematodes can lead to the formation of galls, which causes a disruption of the vascular system of the plant's roots. Root elongation can stop completely, possibly resulting in inadequate supply of water and nutrients provided by the reduced root system, causing foliage chlorosis and/or wilt, as well as stunting of growth, any of which can result in low yield or death. In addition, nematodes can cause physiological effects leading to an increase in the susceptibility of plant roots to bacteria and/or fungi attack, including bacteria and/or fungi the plant would otherwise resist. Such attack can lead to extensive secondary decay and rotting.

The root lesion nematode Pratylenchus brachyurus has become an increasingly important pathogen of soybean. It has a broad host range and is widely distributed in tropical and subtropical regions, especially in Brazil, Africa, and the Southern United States. Pratylenchus brachyurus has become a concern among soybean, corn, and cotton growers in the Brazilian Cerrado region and is considered the main nematode pathogen of soybean in the region. In soybean, this nematode can reduce yields 30 to 50%, with greater damage being observed on sandy soils.

Several methods spanning cultural, biological, and chemical control can be deployed for nematode management. Host-plant resistance, a form of cultural control, has consistently been the most efficacious and cost-effective management method. However, host-plant resistance is not available for many nematode species, especially migratory parasites like Pratylenchus brachyurus. Host-plant resistance can also be limited in its adoption if inheritance is complex (i.e. multiple genes are involved), negative agronomic traits are also conferred, or pest biotypes exist capable of overcoming the resistance. Other cultural control tactics include plant quarantines, crop rotation and tillage. Plant quarantines restrict the importation and movement of plant parts that may harbor or promote the increase of an invasive pest species. Plant quarantines can keep nematodes out of a country or region, but once a nematode species is widely distributed in a country or region, such as P. brachyurus in Brazil, such quarantines are not feasible.

Crop rotation is the practice of altering the growing of multiple crops across seasons in a single location. Crop rotations are an effective management strategy for nematode pests with a limited host range. A nematode's host range is defined as those plants which can support survival and reproduction of the nematode species. Many migratory pests, such as P. brachyurus, have especially broad host ranges, making the rotation to non-hosts impractical. In the case of P. brachyurus in Brazil, all of the major economic crops that are rotated with soybean are suitable hosts. Only a limited number of cover crops such as Crotalaria spectabilis and Crotalaria ochroleuca are non-hosts. However, these cover crops provide no marketable grain or forage yield and therefore represent a cost to plant with no economic return. Leaving a field fallow for a season is another strategy similar to planting non-host cover crops. While leaving a field fallow eliminates the cost of planting a cover crop, it presents inherent risk in potential negative ecological consequences such as soil erosion. Most nematodes can also undergo a quiescent state allowing for long-term survival in fallow fields until a suitable host is planted. In the case of P. brachyurus, survival can be maintained for over 90 days in the absence of a host plant (Ribeiro et al., Heliyon 6: e05075, 2020). Finally, tillage is a potential nematode control tactic, but is not available to those farmers practicing no-till agriculture for soil health reasons. Tillage is the preparation of soil for planting through physical disturbance. Most soybean acres in the Cerrado region of Brazil are farmed under no-till practices (i.e. no mechanical tillage occurs during the growing season or between cropping seasons). Tillage can also have opposite effects on different nematode species. Heterodera glycines (soybean cyst nematode) and P. brachyurus co-occur in many fields in the Cerrado region. Tillage can increase the incidence (Workneh et al., Phytopathology 89: 844-850, 1999), spread (Gavassoni et al., Phytopathology 91: 534-545, 2001), and injury from H. glycines, while two to three rounds of deep tillage are necessary to reduce Pratylenchus spp. populations in some production systems (Khan et al., (2021) Emerging Important Nematode Problems in Field Crops and Their Management. In: Singh K. P., Jahagirdar S., Sarma B. K. (eds) Emerging Trends in Plant Pathology. Springer, Singapore).

Biological control for nematodes is not easily manipulated. Fungal and bacterial seed treatments with nematode-suppressive characteristics have been developed recently. Generally, these seed treatments have several limitations including additional cost, moderate efficacy, variable performance across environments, and short windows of protection, often limited to the early part of the growing season (Dias-Arieira et al., Journal of Phytopathology 166: 722-728, 2018).

Chemical means of controlling plant parasitic nematodes continue to be essential for many crops that lack adequate host plant resistance. However, the activity of chemical agents is often not selective and can have negative effects on non-target organisms, including temporarily disrupting populations of beneficial microorganisms. In recent years the registration of multiple chemical nematicides have been withdrawn, cancelled, or had restrictions placed on their use, limiting the availability of in-furrow nematicides (Fosu-Nyarko and Jones, In Advances in Botanical Research v73 doi: 10.1016/bs.abr.2014.12.012, 2015).

Thus, there exists a need for additional means for controlling nematode populations that injure and/or damage agriculturally-important plants.

### SUMMARY

In one aspect, described herein is a method of suppressing a nematode population in a locus, comprising growing a nematode-resistant plant in a locus, wherein growing the nematode-resistant plant suppresses a nematode population in the locus or maintains the suppression of the nematode population in the locus for a period of time during and/or beyond the growing of the nematode-resistant plant. In some embodiments, the period of time extends to one or more growing seasons subsequent to the growing season in which the nematode-resistant plant was grown. In some embodiments, the methods further comprise growing a secondary plant in the locus at a time subsequent to growing the nematode-resistant plant. In some embodiments, the methods further comprise growing a secondary plant in the locus at a time prior to growing the nematode-resistant plant. In some embodiments, the methods further comprise growing the nematode-resistant plant at the same time as a secondary plant in the locus.

In some embodiments, the secondary plant is a nematode-susceptible plant. In some embodiments, the nematode-susceptible plant is a perennial plant or an annual plant. In some embodiments, the nematode-susceptible plant is a soybean plant, a corn plant, a cotton plant, a canola plant, a sugarcane plant, a potato plant, a wheat plant, a vegetable plant, a rice plant, an alfalfa plant, a barley plant, a sorghum plant, an oat plant, a rye plant, a cassava plant, a sweet potato plant, a sunflower plant, a fruit plant, an orchard plant (such as a fruit or nut tree), an ornamental plant, or a grape vine. In some embodiments, the nematode population is suppressed or maintained at or below the limit of detection. In further embodiments, the secondary plant is Brachiaria and the suppressing of the nematode population is achieved when the number of nematodes is, is about, or is less than about 60 nematodes per gram of root. In some embodiments, the secondary plant is corn and the suppressing of the nematode population is achieved when the number of nematodes is, is about, or is less than about 300 nematodes per gram of root. In still further embodiments, the secondary plant is cotton and the suppressing of the nematode population is achieved when the number of nematodes is, is about, or is less than about 60 nematodes per gram of root. In some embodiments, the secondary plant is sorghum and the suppressing of the nematode population is achieved when the number of nematodes is, is about, or is less than about 250 nematodes per gram of root. In further embodiments, the suppressing of the nematode population in the locus in which the secondary plant is grown is achieved when there is, is about, or is at least about a 5% reduction in the number of nematodes per gram of root relative to the number of nematodes per gram of root in a comparable locus to the locus in which the secondary plant is grown.

In another aspect, described herein is a method of nematode management for a locus, comprising growing a nematode-resistant plant in a locus in a first growing season, wherein growing the nematode-resistant plant in the first growing season suppresses a nematode population in the locus or maintains the suppression of the nematode population in the locus; growing a nematode-susceptible plant in the locus in the same or a subsequent growing season; and achieving improved health and/or yield of the nematode-susceptible plant compared to the health and/or yield expected if the nematode population was not suppressed.

In some embodiments, the nematode-resistant plant expresses a Cry protein, for example and without limitation a nematicidal Cry protein. In some embodiments, the nematode-resistant plant is a soybean plant, a corn plant, a cotton plant, a canola plant, a sugarcane plant, a sugar beet plant, a potato plant, a wheat plant, a rice plant, an alfalfa plant, a barley plant, a sorghum plant, an oat plant, a rye plant, a cassava plant, a sweet potato plant, a sunflower plant, a vegetable plant, a fruit plant, an ornamental plant, an orchard plant (such as a fruit or nut tree), or a grape vine.

In some embodiments, the nematode is a nematode species selected from Pratylenchus spp. nematode populations, e.g., Pratylenchus brachyurus, Meloidogyne spp., Heterodera spp., e.g. Heterodera glycines, Globodera spp., Rotylenchulus reniformis, Helicotylenchus spp., e.g. Helicotylenchus dihystera, Scutellonema brachyurus, Tubixaba tuxaua, or Aphelencoides besseyi. In some embodiments, the Pratylenchus spp is Pratylenchus brachyurus.

In some embodiments, the improved health of the nematode-susceptible plant comprises one or more of the following: improved root development (e.g., improved root or root hair growth); improved yield; faster emergence; improved plant stress management including increased stress tolerance and/or improved recovery from stress; increased mechanical strength; improved drought resistance; reduced fungal, bacterial and/or viral disease infection; or any combination thereof.

Also provided are methods of protecting a nematode-susceptible plant from nematode injury or damage comprising growing a nematode-resistant plant in a locus at least one growing season before planting the nematode-susceptible plant; and growing a nematode-susceptible plant in the locus at least one growing season subsequent to growing the nematode-resistant plant. In some embodiments, protecting the nematode-susceptible plant from nematode damage comprises an increase in yield from harvested nematode-susceptible plant material, or in money made from the sale of harvested nematode-susceptible plant material.

Also provided are methods of nematode management for a locus, the method comprising growing a nematode-resistant plant in a locus; and growing a nematode-susceptible plant in the locus at a time subsequent to growing the nematode-resistant plant. In some embodiments, the nematode-susceptible plant is a perennial plant. In some embodiments, the nematode-susceptible plant is an annual plant.

In another aspect, the disclosure provides methods of nematode management for a locus, the method comprising growing a nematode-resistant plant in a locus at the same time as a nematode-susceptible plant. In some embodiments, the nematode-susceptible plant is a perennial plant. In some embodiments, the nematode-susceptible plant is an annual plant.

In another aspect, the disclosure provides methods of nematode management for a locus comprising planting a nematode-susceptible plant in a locus, wherein the nematode-susceptible plant is an annual plant or a perennial plant; and growing a nematode-resistant plant in the locus at a time following the planting of the nematode-susceptible plant. In some embodiments, the nematode-susceptible plant is a vegetable plant, a fruit plant, an orchard plant, an ornamental plant, or a grape vine.

In another aspect, the disclosure provides a locus having a suppressed nematode population density, wherein the suppressed nematode population density is achieved by the methods described herein. A nematode-resistant plant grown in the locus, plant material harvested from the plant and seeds produced by the plant are also provided, as is a locus that does not (i) require tillage; (ii) require a cover crop; or (iii) need to lie fallow one growing season per year or per crop rotation. In some embodiments, the locus provides one or more of the following benefits: a. the locus does not does not need to lie fallow one growing season per crop rotation cycle; b. the locus does not need to be tilled one growing season per crop rotation cycle; and c. the locus does not need to be planted with a cover crop. In some embodiments, the suppressed nematode population density is, is about, or is less than about 250, 200, 150, 100, 50, 20, or 10 nematodes per gram (g) of root.

The methods and systems described herein provide benefit and value in a crop rotation system by (i) enabling increased use of a locus that does not need to lie fallow, be tilled, or grow a cover crop during at least one growing season in a crop rotation cycle in an effort to control nematodes and (ii) enabling more successful use of a locus by suppressing the nematode population in the locus, resulting in successful growth and increased yield of nematode-susceptible plants of intrinsic value. A nematode-susceptible plant grown in the locus, plant material harvested from the nematode-susceptible plant (e.g., cotton lint and cotton fiber in the case of cotton plants) and seeds produced by the nematode-susceptible plant are also provided.

In another aspect, the disclosure provides a system for increased use of a locus, the system comprising growing a nematode-resistant plant in a locus in a first growing season; and growing a nematode-susceptible plant in the locus in a subsequent growing season. In some embodiments, each of the nematode-resistant plant and the first nematode-susceptible plant is an intrinsic value crop plant. In some embodiments, over the course of a subsequent growing season the locus is not fallowed for a growing season. In some embodiments, no cover plant or cover crop is grown during the subsequent growing season. In some embodiments, the nematode-resistant plant, a first nematode-susceptible plant, and a second nematode-susceptible plant are grown in three consecutive growing seasons. In some embodiments, each of the nematode-resistant plant, the first nematode-susceptible plant and the second nematode-susceptible plant is an intrinsic value crop plant.

In yet another aspect, the disclosure provides a method for improving a crop rotation system, the method comprising growing a nematode-resistant plant in a locus during a first growing season; and growing a nematode-susceptible plant in the locus during a subsequent growing season, wherein growing the nematode-resistant plant in the locus during the first growing season results in a suppression of the nematode population in the locus that enables or improves the growth of the nematode-susceptible plant in the subsequent growing season. In some embodiments, the improved crop rotation system may further include one or more of the following:
a. use of the locus for at least one additional growing season per year;
b. decreased tillage of the locus;
c. decreased treatment of the nematode-susceptible crop seed with nematicides;
d. decreased treatment area of the locus with nematicides prior to or during the growing season for the nematode-susceptible plant;
e. decreased rate of nematicide applied to the nematode-susceptible plant and/or the locus prior to or during the growing season for the nematode-susceptible plant;
f. decreased number of applications of nematicides made to the nematode-susceptible plant and/or the locus during the growing season;
g. increased usability of the locus;
h. increased value of the locus;
i. improved sustainable agricultural practices and/or
j. increased yield from the nematode-susceptible crop.

In another aspect, the disclosure provides a method of nematode management for a locus, the method comprising: growing a nematode-resistant plant in a locus during a growing season; and growing a perennial plant in the locus before, during, and/or after the growing season, wherein growing the nematode-resistant plant in the locus results in a suppression of the nematode population in the locus that enables or improves the growth of the perennial plant.

In some aspects, the disclosure provides methods for marketing a crop rotation system, the method comprising: promoting use of a nematode-resistant plant during a first growing season; and promoting use of a nematode-susceptible plant in the locus during the same or a subsequent growing season, wherein growing the nematode-resistant plant in the locus during the first growing season results in a suppression of the nematode population in the locus that enables or improves the growth of the nematode-susceptible plant in the subsequent growing season. In some embodiments, the subsequent growing season is immediately adjacent to the first growing season.

In further aspects, the disclosure provides marketing materials directed to a crop rotation system of growing a nematode-resistant plant during a first growing season in coordination with or followed by growing a nematode-susceptible plant in the locus during a subsequent growing season, wherein growing the nematode-resistant plant in the locus during the first growing season results in a suppression of the nematode population in the locus that enables or improves the growth of the nematode-susceptible plant. In some embodiments, the marketing materials are directed to promoting the crop rotation system.

In some aspects, methods of nematode management for a locus are provided, the methods comprising: growing a nematode-resistant plant in a locus prior to, at the same time as, or after growing a secondary crop in the locus. In some embodiments, the nematode-resistant plant expresses a nematicidal Cry14Ab protein having at least 95, 96, 97, 98, or at least 99% sequence identity to SEQ ID NO. 1. In some embodiments, the nematode-resistant plant comprises elite event EE-GM5. In further embodiments, the nematode-resistant plant comprises elite event EE-GM4. In any of the aspects or embodiments of the disclosure, the nematode-resistant plant expresses Bacillus thuringiensis toxin Cry14Ab-1. In any of the aspects or embodiments of the disclosure, the nematode-resistant plant comprises elite event EE-GM5. In any of the aspects or embodiments of the disclosure, the nematode-resistant plant comprises elite event EE-GM4.

### BRIEF DESCRIPTION OF THE FIGURES

FIG. 1 is a graph showing the effect of GMB151 soybean on Pratylenchus brachyurus population densities. The population density of Pratylenchus brachyurus was estimated for the GMB151 Homozygous and Nullizygous soybean lines at the three anticipated high-pressure sites and the Sinop2 site, which had higher P. brachyurus population densities than anticipated. The GMB151 transgenic soybean trait significantly reduced P. brachyurus populations. The 95% confidence interval is represented by the error bars about the estimated difference between the Homozygous and Nullizygous lines.

FIG. 2 is a graph showing the soybean yield difference between the GMB151 Homozygous and Nullizygous line at each of the research sites. Research sites are arranged from lowest to highest Pratylenchus brachyurus population density. Error bars about the estimated yield differences represent the 95% confidence intervals. The GMB151 trait did not affect yield at the three locations with the lowest P. brachyurus population densities. At the three sites with the highest P. brachyurus population densities, GMB151 significantly improved soybean yield by an average of 7.0 bu/acre or 21%.

FIG. 3 provides a diagram of field trial plantings described in Example 3. Soybean was planted in the safra (summer or first crop) soybean season. Five replications were planted per treatment in a randomized complete block design. Safrinha (winter or second crop) cotton and corn trials were planted after soybean harvest. Safrinha crop trials were planted on top of the soybean trial ground with safrinha crop rows running perpendicular to the harvested safra soybean rows. Safrinha crops trial designs also constituted a randomized complete block design, but with only three replications per crop.

FIG. 4 is a graph showing the amount of Pratylenchus brachyurus control provided by GMB151 soybean in 2019/20 Brazilian growing season at the Rio Verde, Goias field site. Asterisks represent significant differences at P<0.05.

FIG. 5 is a graph showing safra soybean and safrinha corn yield for the 2019/20 growing season at the Rio Verde, Goias field site and gross revenue.

FIG. 6 is a graph showing, Pratylenchus brachyurus control (as amount of Pratylenchus brachyurus per gram of root in safrinha crops) provided by GMB151 soybean in 2019/20 Brazilian growing season across all field trial sites.

FIG. 7 is a graph showing safrinha corn and cotton yields for the 2019/20 growing season across all field test sites.

FIG. 8 provides an exemplary crop rotation system.

FIG. 9 is a graph showing the effect of GMB151 soybean on Pratylenchus brachyurus population densities. The population density of Pratylenchus brachyurus was estimated for the GMB151 Homozygous and Nullizygous soybean lines at the 49 trial sites. The GMB151 transgenic soybean trait significantly reduced P. brachyurus populations. The standard error of the mean is represented by the error bars about the mean of the Homozygous and Nullizygous lines.

FIG. 10 is a graph showing the soybean yield difference between the GMB151 Homozygous and Nullizygous lines across the 48 trial sites taken to yield. Error bars depict the standard error about the treatment mean. The GMB151 trait significantly improved soybean yield by an average of 4.2 bu/acre or 9%.

FIG. 11 is a graph showing alternative nematode management tools and their effect on Pratylenchus brachyurus population densities in the safra soybean crop. Nematicide seed treatments, and in-furrow nematicides were applied to both GMB151 and Null soybean. Significantly greater control was provided by GMB151 soybean than by either seed treatment or in-furrow nematicides.

FIG. 12 is a graph showing the efficacy of the nematode cultural control tactic of tillage and its effect on Pratylenchus brachyurus population densities in comparison to the GMB151 soybean event. The GMB151 soybean event provided significantly greater control of Pratylenchus brachyurus than conventional tillage in the safra soybean crop.

FIG. 13 is a graph showing Pratylenchus brachyurus control (as amount of Pratylenchus brachyurus per gram of root in safrinha crops) provided by GMB151 soybean in safrinha crop field trials.

### DETAILED DESCRIPTION

Plant-parasitic nematodes are capable of attacking multiple species of cultivated plants (defined herein as “crops” or “agronomic crops”) in a farming rotation or crop rotation system and can build up large population densities over time. Cultivated plants that are viewed by farmers to be economically profitable (“cash crops”) or are otherwise valuable or useful to a farmer (such as livestock feed) and all of which are “intrinsic value” crops, as well as profitless plants or crops cultivated solely to protect a field from erosion or to provide other soil health benefits (“cover crops”), are nearly all susceptible to a greater or lesser degree to nematode injury. Certain plants may be either an intrinsic value crop or a profitless crop, depending on how they are used. One such plant is Brachiaria, also known as signal grass. Signal grass may be grown as either a grazing crop (and thus an intrinsic value crop) or a profitless cover crop when not used for grazing. Notably, cash crops such as soybean, cotton, corn, wheat, sugarcane, potatoes, sugar beets, rice, alfalfa, barley, sorghum, oats, rye, cassava, sweet potatoes, sunflower, vegetables, fruit plants, fruit trees, nut trees, ornamental plants, grape vines and canola, as non-limiting examples, are susceptible to nematodes, and injury or damage caused by nematodes can reduce yield significantly, leading to a reduction in the farmer's income. Farmers must identify effective management options for controlling nematode pest injury and damage in each crop they grow.

In addition, farmers may have to adjust their crop rotation or crop succession order to avoid growing multiple susceptible crops in consecutive growing seasons. This problem can be challenging when farmers are presented with nematodes with wide host ranges capable of reproducing on many agronomic crops. In worst-case scenarios, a farmer may be forced to forgo planting an economically profitable cash crop in favor of planting a profitless cover crop or even worse leaving a field fallow due to the exceptionally wide host range of some nematodes which include even most cover crops. Even leaving a field fallow provides only limited relief to the farmer as most nematodes can survive extended period of times (>9 months) without a host plant. The nematode pest can simply exist in a dormant state until the farmer plants a susceptible crop.

Farmers suffer additionally from managing the agronomic challenges presented by nematodes, considering that such challenges must be met in the bigger context of each fanner's need to adhere to other requirements meant to protect the overall environment and address ecological issues such as soil erosion. Currently, there are cases in which nematode-management practices and other agronomic practices and requirements are contradictory, leaving farmers to make difficult choices. For example, Pratylenchus spp. can be managed through tillage performed multiple times per year. However, tillage can promote soil erosion, water loss, organic carbon loss, and generally be less favorable for crop yields. In addition, even in situations where a farmer may be willing to plant an unprofitable cover crop for nematode management, the cover crops that limit nematode reproduction may not be the same cover crops that would provide larger soil health benefits such as increasing organic carbon, (Amorim et al., Journal of Agricultural Science 11: 333-340, 2019). Methods of nematode management that include approaches that suppress or prevent an increase in a nematode population in a locus while limiting interference with the growth of agronomic crops are of greatest benefit to the farmer.

The present disclosure is based on the discovery that the benefits of the nematicidal activity of a nematode-resistant plant grown in a locus (e.g., a field or plot) extend beyond the period of time during which the nematode resistant plant is grown. Growing the nematode-resistant plant in a locus suppresses a nematode population in the locus or maintains the suppression of a nematode population in the locus, wherein the suppression of the nematode population or the maintenance of the suppression of the nematode population lasts for a period of time beyond the growing of the nematode-resistant plant. Accordingly, in some aspects, the disclosure provides methods of nematode management for a locus, the method comprising: growing a nematode-resistant plant in a field prior to, at the same time as, or after growing a secondary plant in the locus.

In some embodiments, the benefits of the nematicidal activity of a nematode-resistant plant grown in a locus (e.g., a field or plot) in a first growing season, wherein such nematicidal activity protects the nematode-resistant plant from nematode damage and injury, also extends nematode protection to any other plant (e.g., a cash crop that does or does not include nematode-resistant plants) by simply growing the other plant in the same locus in the same or a subsequent growing season. The nematode-resistant plant can be grown on a regular basis (e.g., consecutive growing seasons, every other growing season, every third growing season, every fourth growing season, every fifth growing season, etc.) to reduce or control the overall population density of the nematode in a particular locus. In some embodiments, the nematode-resistant plant is grown every growing season. In some embodiments, the nematode-resistant plant is grown in the growing season that is immediately adjacent to the growing season of a secondary plant (e.g., a nematode-susceptible plant). Thus, the nematode-resistant plant not only protects itself from nematode injury, but also protects any plant, including a nematode-susceptible plant, that is grown in the same locus at the same time or at a later time.

The term “locus” or “loci” as used herein refers to a location (or locations) suitable for growing a plant. Exemplary loci include, but are not limited to, a pot or other container, a green house or other contained location, a field, a hill, any plot of land, an orchard, a vineyard, or other environment suitable for growing plants.

The term “nematode-resistant plant” as used herein refers to a plant that expresses a nucleic acid that results in impairment of the movement, feeding, development, reproduction, or other functions of a nematode if the nematode is in contact with the plant. In some embodiments, the nematode-resistant plant has been manipulated or is derived from a plant that has been manipulated via a molecular biology technique to express such nucleic acid. Without limitation, one example of nematode impairment is when a nematode is killed by ingestion of a part of the nematode-resistant plant. In some embodiments, nematode-resistant plants include, but are not limited to, a soybean plant, a corn plant, a cotton plant, a canola plant, a sugarcane plant, a sugar beet plant, a potato plant, or a wheat plant, a rice plant, an alfalfa plant, a barley plant, a sorghum plant, an oat plant, a rye plant, a cassava plant, a sweet potato plant, a sunflower plant, a vegetable plant, a fruit plant, an ornamental plant, an orchard plant (such as a fruit or nut tree), or a grape vine.

The term “nematode-susceptible plant” as used herein refers to a plant that does not express a nucleic acid that results in impairment of the movement, feeding, development, reproduction or other functions of the nematode if the nematode is in contact with the plant. One of ordinary skill in the art understands that any plant can be a nematode-resistant or nematode-susceptible plant, depending on whether the plant expresses a nucleic acid that results in impairment of the movement, feeding, development, reproduction, or other functions of a nematode if the nematode is in contact with the plant. In some embodiments, the nematode-resistant plant has been manipulated or is derived from a plant that has been manipulated via a molecular biology technique to express such nucleic acid. In some embodiments, nematode-susceptible plants include, but are not limited to, a soybean plant, a corn plant, a cotton plant, a canola plant, a sugarcane plant, a potato plant, a wheat plant, a vegetable plant, a rice plant, an alfalfa plant, a barley plant, a sorghum plant, an oat plant, a rye plant, a cassava plant, a sweet potato plant, a sunflower plant, a fruit plant, an orchard plant (such as a fruit or nut tree), an ornamental plant, or a grape vine.

The present disclosure is also based on the discovery that the growth of a nematode-resistant plant in a locus results in a lasting suppression of the nematode population density in the locus. Nematode suppression can be measured in a number of ways including, but not limited to, ascertaining the nematode population density in a locus, in plant roots grown in the locus, or in another meaningful area.

### Methods

The present disclosure is directed to a method of suppressing a nematode population in a locus, the method comprising: growing a nematode-resistant plant in a locus, wherein growing the nematode-resistant plant suppresses a nematode population in the locus for a period of time during and beyond the growing of the nematode-resistant plant. In some embodiments, the period of time extends to one or more growing seasons subsequent to the growing season in which the nematode-resistant plant was grown.

In some embodiments, the method further comprises growing a secondary plant (which can be any plant) in the locus at a time subsequent to growing the nematode-resistant plant. In some embodiments, the method further comprises growing a secondary plant in the locus at a time prior to growing the nematode-resistant plant. In some embodiments, the method further comprises growing the nematode-resistant plant at the same time as a secondary plant in the locus. In some embodiments, the secondary plant is a nematode-susceptible plant. In some embodiments, the nematode-resistant plant is grown in a row that is adjacent to the row in which the secondary plant is grown. In some embodiments, the nematode-resistant plant is grown in the same row as the row in which the secondary plant is grown. In some embodiments, the nematode-resistant plant is grown both in a row that is adjacent to the row in which the secondary plant is grown and in the same row as the row in which the secondary plant is grown. The disclosure contemplates any configuration known in the art for growing the nematode-resistant plant and the secondary plant (e.g., grown in the same or adjacent rows; grown in narrow versus wide row width; grown on the edges of a field vs. in the center of a field; grown intermixed with no distinct rows). In some embodiments, the secondary plant is a nematode-susceptible plant.

The present disclosure is also directed to a method of suppressing a nematode population in a locus, the method comprising: growing a nematode-resistant plant in a locus, wherein growing the nematode-resistant plant suppresses or maintains the suppression of an already-suppressed nematode population in the locus for a period of time during and/or beyond the growing of the nematode-resistant plant. In some embodiments, the period of time extends to one or more growing seasons subsequent to the growing season in which the nematode-resistant plant was grown. For example, a nematode-resistant plant can be grown in a locus in which a nematode-resistant plant has been previously grown and/or in which some other mechanism for suppressing the nematode population has been previously utilized.

The present disclosure is also directed to a method of suppressing a nematode population, the method comprising: growing a nematode-resistant plant in a locus in a first growing season, wherein the growing of the nematode-resistant plant results in suppression of the nematode population in the locus, and growing a secondary plant (which can be any plant) in the same locus in the same or a subsequent growing season (e.g., a second, third, fourth, or fifth growing season), wherein the secondary plant exhibits less nematode injury than would be seen if the nematode-resistant plant had not been grown in the locus in the first growing season. In some embodiments, the subsequent growing season is the immediately adjacent growing season.

In some embodiments, the nematode population shows little or no resurgence during the subsequent growing season. The first growing season may be any growing season throughout the year. Nematode-resistant plants may be grown during one or more growing seasons per year, including during consecutive growing seasons. In some embodiments, the nematode population of a locus is already suppressed and the growing of the nematode-resistant plant in the locus maintains the suppression of the nematode population. The growth of the nematode-resistant plant does not need to be for a particular amount of time or to a particular stage of development, so long as the nematode population in the locus is suppressed or suppression of the nematode population in the locus is maintained. Suppression of a nematode population may be maintained by growing a nematode-resistant plant in a locus that has previously hosted a nematode-resistant plant that suppressed the nematode population, wherein the maintained suppression of the nematode population may be indicated by the presence of fewer nematodes in the locus than in the same or a comparable locus. Suppression of a nematode population or maintenance of suppression of a nematode population may be indicated by the presence of fewer nematodes in a locus than in the same or a comparable locus if no nematode management measures have been taken, including growing a nematode-resistant plant. Alternatively, or in addition, suppression of a nematode population or maintenance of suppression of a nematode population can mean that growth of the nematode-resistant plant in a locus achieves and/or maintains a level of nematodes at the locus that is at or below the limit of detection.

In some embodiments, suppression of a nematode population or maintenance of suppression of a nematode population is indicated by the presence of fewer nematodes in a locus than in the same or a comparable locus if no nematode management measures have been taken, including growing a nematode-resistant plant. In some embodiments, a comparable locus is a locus in which a nematode-resistant plant has never been grown. In some embodiments, a comparable locus is a locus in which a nematode-resistant plant has been grown prior to, but not during, the current crop rotation cycle.

In some embodiments, suppression of a nematode population or maintenance of suppression of a nematode population when the secondary plant is Brachiaria is achieved when the number of nematodes is, is about, or is less than about 60 nematodes per gram of root. In further embodiments, suppression of a nematode population when the secondary plant is Brachiaria is achieved when the number of nematodes is, is about, or is less than about 50, 40, 30, 20, 10, or 5 nematodes per gram of root. In further embodiments, suppression of a nematode population when the secondary plant is Brachiaria is achieved when the number of nematodes is or is about 5-60, 10-60, 20-60, 5-50, 10-50, 20-50, 5-40, 5-30, 5-20, 10-40, 10-30, 10-20, 20-40, or 20-30 nematodes per gram of root.

In some embodiments, suppression of a nematode population or maintenance of suppression of a nematode population when the secondary plant is corn is achieved when the number of nematodes is, is about, or is less than about 300 nematodes per gram of root. In further embodiments, suppression of a nematode population when the secondary plant is corn is achieved when the number of nematodes is, is about, or is less than about 250, 200, 150, 100, 50, 20, 10, or 5 nematodes per gram of root. In further embodiments, suppression of a nematode population when the secondary plant is corn is achieved when the number of nematodes is or is about 5-300, 5-250, 5-200, 5-150, 5-100, 5-50, 10-300, 10-250, 10-200, 10-150, 10-100, 10-50, 50-300, 50-250, 50-200, 50-150, or 50-100 nematodes per gram of root.

In some embodiments, suppression of a nematode population or maintenance of suppression of a nematode population when the secondary plant is cotton is achieved when the number of nematodes is, is about, or is less than about 60 nematodes per gram of root. In further embodiments, suppression of a nematode population when the secondary plant is cotton is achieved when the number of nematodes is, is about, or is less than about 50, 40, 30, 20, 10, or 5 nematodes per gram of root. In further embodiments, suppression of a nematode population when the secondary plant is cotton is achieved when the number of nematodes is or is about 5-60, 10-60, 20-60, 5-50, 10-50, 20-50, 5-40, 5-30, 5-20, 10-40, 10-30, 10-20, 20-40, or 20-30 nematodes per gram of root.

In some embodiments, suppression of a nematode population or maintenance of suppression of a nematode population when the secondary plant is sorghum is achieved when the number of nematodes is, is about, or is less than about 250 nematodes per gram of root. In further embodiments, suppression of a nematode population when the secondary plant is sorghum is achieved when the number of nematodes is, is about, or is less than about 200, 150, 100, 50, 20, 10, or 5 nematodes per gram of root. In further embodiments, suppression of a nematode population when the secondary plant is sorghum is achieved when the number of nematodes is or is about 5-250, 5-200, 5-150, 5-100, 5-50, 10-250, 10-200, 10-150, 10-100, 10-50, 50-250, 50-200, 50-150, or 50-100 nematodes per gram of root.

In some embodiments, suppression of a nematode population or maintenance of suppression of a nematode population in a locus in which a secondary plant is grown is achieved when there is, is about, or is at least about a 5% reduction in the number of nematodes per gram of root relative to the number of nematodes per gram of root in a comparable locus to the locus in which the secondary plant is grown. In further embodiments, suppression of a nematode population in a locus (e.g., a locus in which a secondary plant is grown) is achieved when there is, is about, or is at least about a 5%, 10%, 20%, 25%, 30%, 40%, 50%, 60%, 70%, or 80% reduction in the number of nematodes per gram of root relative to the number of nematodes per gram of root in a comparable locus if no nematode management measures have been taken, including growing a nematode-resistant plant. In some embodiments, a comparable locus is a locus in which a nematode-resistant plant has never been grown. In some embodiments, a comparable locus is a locus in which a nematode-resistant plant has been grown prior to, but not during, the current crop rotation cycle. In any of the aspects or embodiments of the disclosure, determination of the number of nematodes per gram of root is performed by a person of ordinary skill in the art.

The present disclosure is directed to a method of suppressing a nematode population, the method comprising: growing a nematode-resistant plant in a locus in a first growing season, wherein the growing of the nematode-resistant plant results in suppression of the nematode population in the locus, and growing a nematode-susceptible plant in the same locus in the same or a subsequent growing season (e.g., a second, third, fourth, or fifth growing season), wherein the nematode-susceptible plant exhibits less nematode injury than would be seen if the nematode-resistant plant had not been grown in the locus in the first growing season. In some embodiments, the nematode population shows little or no resurgence during the subsequent growing season.

In some embodiments, the method comprises growing the nematode-resistant plant in a locus in a first growing season, growing a first nematode-susceptible plant in the locus in a subsequent growing season (e.g., in a second, third, fourth, or other growing season subsequent to growing the nematode-resistant plant in the locus), and growing a second nematode-susceptible plant in the locus subsequent to growing the first nematode-susceptible plant. A third, fourth, fifth, etc. nematode-susceptible plant may be grown in the locus in subsequent growing seasons.

In some embodiments, a nematode-resistant plant is grown in the locus prior to growing a first nematode-susceptible plant. One of skill in the art understands that no particular order of planting is required once nematode suppression is achieved in a locus.

In some embodiments, the nematode-resistant plant is a monocot. In some embodiments, the nematode-resistant plant is a dicot. In some embodiments, the nematode-resistant plant is an annual plant. In some embodiments, the nematode-resistant plant is a perennial plant.

In some embodiments, the nematode-resistant plant is a soybean plant, a corn plant, a cotton plant, a canola plant, a sugarcane plant, a sugar beet plant, a potato plant, a wheat plant a rice plant, an alfalfa plant, a barley plant, a sorghum plant, an oat plant, a rye plant, a cassava plant, a sweet potato plant, a sunflower plant, a vegetable plant, a fruit plant, an ornamental plant, an orchard plant (such as a fruit or nut tree), or a grape vine.

In some embodiments, the nematode-resistant plant is a soybean plant and the soybean plant is grown in a locus in a first growing season and the nematode-susceptible plant is a soybean plant, a corn plant, a cotton plant, a canola plant, a sugarcane plant, a sugar beet plant, a potato plant, a wheat plant, a rice plant, an alfalfa plant, a barley plant, a sorghum plant, an oat plant, a rye plant, a cassava plant, a sweet potato plant, a sunflower plant, a vegetable plant, a fruit plant, an orchard plant (such as a fruit or nut tree), an ornamental plant, or a grape vine grown in the same field or other locus in a second growing season, and the nematode-resistant soybean plant is grown in a third growing season.

In some embodiments, the nematode-resistant plant is a soybean plant and the soybean plant is grown in a locus in a first growing season and the nematode-susceptible plant is a soybean plant, a corn plant, a cotton plant, a canola plant, a sugarcane plant, a sugar beet plant, a potato plant, a wheat plant, a rice plant, an alfalfa plant, a barley plant, a sorghum plant, an oat plant, a rye plant, a cassava plant, a sweet potato plant, a sunflower plant, a vegetable plant, a fruit plant, an orchard plant (such as a fruit or nut tree), an ornamental plant, or a grape vine and is grown in the same locus in the same or a second growing season, and optionally a nematode-susceptible plant is grown in the same locus in a third growing season, wherein the nematode-susceptible plant is a soybean plant, a corn plant, a cotton plant, a canola plant, a sugarcane plant, a sugar beet plant, a potato plant, a wheat plant, a rice plant, an alfalfa plant, a barley plant, a sorghum plant, an oat plant, a rye plant, a cassava plant, a sweet potato plant, a sunflower plant, a vegetable plant, a fruit plant, an orchard plant (such as a fruit or nut tree), an ornamental plant, or a grape vine. For annual plants, the seasonal order of the planting doesn't matter, so long as a nematode-resistant plant is grown in the locus sometime prior to or simultaneously with the planting of an annual plant, and so long as the nematode population in the locus becomes or remains suppressed enough to provide a benefit to the annual plant.

In some embodiments, a nematode-susceptible plant is grown in a locus with a nematode-resistant plant in the locus during the same growing season or at the same time. In some embodiments, the nematode-susceptible plant may be a perennial plant including, but not limited to, a vegetable plant, a fruit plant, an orchard plant (e.g., a fruit tree or a nut tree), an ornamental plant, or a grape vine. In this way, a nematode-resistant plant can be used to protect perennials from nematode injury. In some embodiments, a nematode-resistant plant is grown in a locus prior to planting a perennial plant to suppress the nematode population in the locus prior to planting the perennial plant. In some embodiments, a nematode-resistant plant is grown both before a perennial plant is planted and is grown at the same time as or with the perennial, for example on a yearly basis if the nematode-resistant plant is an annual plant. In some embodiments, a nematode-resistant plant is grown in a locus already containing a perennial plant, wherein the perennial plant obtains a benefit from the addition of the nematode-resistant plant to the locus. For perennial plants, the seasonal order of the planting does not matter, so long as a nematode resistant plant is grown in the locus prior to, concurrently with, or after the planting of the perennial plant, and so long as the nematode population in the locus becomes or remains suppressed enough to provide a benefit to the perennial plant.

Also provided is a method of protecting a nematode-susceptible plant from nematode damage or nematode injury comprising growing a nematode-resistant plant in a locus at least one growing season prior to growing the nematode-susceptible plant; and growing the nematode-susceptible plant in the locus in the same or at least one growing season subsequent to the growing season for the nematode-resistant plant. The term “nematode injury” as used herein refers to physical harm or destruction caused to the plant by the nematode. The term “nematode damage” as used herein refers to monetary loss caused to the marketable commodity by the nematode.

Methods for increasing yield of a nematode-susceptible plant are also provided. The methods comprise growing a nematode-resistant plant in a field for a first growing season; and growing a nematode-susceptible plant in the field during the same or a subsequent growing season. Growing the nematode-susceptible plant in the same growing season or a season subsequent to growth of the nematode-resistant plant increases the yield of the nematode-susceptible plant compared to the yield of the nematode-susceptible plant grown in the same or a comparable locus if no nematode management measures have been taken, including growing a nematode-resistant plant. In some embodiments, the increased yield of the nematode-susceptible plant is compared to the yield of the nematode-susceptible plant grown in the same or a comparable locus in which the nematode-resistant crop has not been grown during the most recent crop rotation cycle. In some embodiments, a comparable locus is a locus in which a nematode-resistant plant has never been grown. In some embodiments, a comparable locus is a locus in which a nematode-resistant plant has been grown prior to, but not during, the current crop rotation cycle.

As used herein, the term “yield” of the plant refers to the quality and/or quantity of biomass produced by the plant. By “biomass” is intended any measured plant product. An increase in biomass production is any improvement in the yield of the measured plant product. Increasing plant yield has several commercial applications. For example, increasing plant leaf biomass may increase the yield of leafy vegetables for human or animal consumption. Additionally, increasing leaf biomass can be used to increase production of plant-derived pharmaceutical or industrial products. In some embodiments, growing the nematode-susceptible plant subsequent to growing the nematode-resistant plant increases the yield of the nematode-susceptible plant by at least 1% (or at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%) compared to a nematode-susceptible plant that was grown in the same or a comparable locus if no nematode management measures have been taken, including growing a nematode-resistant plant. In some embodiments, a comparable locus is a locus in which a nematode-resistant plant has never been grown. In some embodiments, a comparable locus is a locus in which a nematode-resistant plant has been grown prior to, but not during, the current crop rotation cycle.

In various embodiments, growing a nematode-susceptible plant in a growing season concurrent with or subsequent to the growth of a nematode-resistant plant results in, for example, the following benefits for the nematode-susceptible plant: improved root development (e.g., improved root or root hair growth); improved yield; faster emergence; improved plant stress management including increased stress tolerance and/or improved recovery from stress; increased mechanical strength; improved drought resistance; reduced fungal, bacterial and/or viral disease infection; and/or improved plant health compared to a nematode-susceptible plant that was grown in the same or a comparable locus if no nematode management measures have been taken, including growing a nematode-resistant plant. In some embodiments, a comparable locus is a locus in which a nematode-resistant plant has never been grown. In some embodiments, a comparable locus is a locus in which a nematode-resistant plant has been grown prior to, but not during, the current crop rotation cycle. Combinations of any of these benefits can also be obtained.

In various embodiments, growing a plant in a growing season subsequent to or concurrent with the growth of a nematode-resistant plant in a particular locus can enable growth of plants that are not amenable to becoming nematode-resistant plants due to technical, cultural, and/or regulatory reasons. Non-limiting examples of such plants that can be used in the invention include fruit or nut trees, ornamental plants, grape vines, or wheat in Australia.

The present invention is applicable to both migratory and sedentary nematode species. Further, there are emerging species of nematodes in both the migratory and sedentary categories that may also be controllable by the present invention. In some embodiments, the nematode is from a nematode population, particularly Pratylenchus spp. nematode populations, e.g., Pratylenchus brachyurus, Meloidogyne spp., Heterodera spp., e.g. Heterodera glycines, Globodera spp., Rotylenchulus reniformis, Helicotylenchus spp., e.g. Helicotylenchus dihystera, Scutellonema brachyurus, Tubixaba tuxaua, or Aphelencoides besseyi.

A locus, such as a field or plot, having a suppressed nematode population density (e.g., nematodes per volume of soil) or reduced nematode numbers (e.g., nematodes per gram of root), wherein the suppressed nematode population density or reduced nematode numbers are achieved by the methods described herein are also contemplated. Measurements of a suppressed nematode population density or reduced nematode numbers in a locus are in a practical sense interchangeable, as both are an indication of the nematode presence in the locus, with the distinction being only in the method used to measure the nematode presence. A suppressed nematode population can be measured in any number of ways, none of which are limiting to the invention. In various embodiments, the suppressed nematode population density is, is about, or is less than about 250, 200, 150, 100, 50, 20, or 10 nematodes per gram (g) of root.

### Nematicidal Nucleic Acids, Nematicidal Proteins, and Nematode-Resistant Plants

The methods described herein describe the use of a plant that expresses a nematicidal nucleic acid. In some embodiments, the methods described herein involve use of a plant manipulated by molecular biology techniques via transformation of organisms or use of organisms comprising a heterologous nucleotide sequence encoding a nematicidal protein. There are a number of molecular biology techniques that can be used to enable a plant to express a nematicidal nucleic acid, and the technical approach used to obtain a nematode-resistant plant is not limiting to the methods herein.

The terms “nematicidal nucleic acid” and “nematicidal protein” as used herein refer to a toxin that has activity against one or more nematode pests, including, but not limited to, Meloidogyne spp., Rotylenchulus reniformis, Helicotylenchus dihystera, Scutellonema brachyurus, Tubixaba tuxaua, Aphelencoides besseyi, and Pratylenchus spp., including Pratylenchus alleni, Pratylenchus brachyurus, Pratylenchus coffeae, Pratylenchus crenatus, Pratylenchus dulscus, Pratylenchus fallax, Pratylenchus flakkensis, Pratylenchus goodeyi, Pratylenchus hexincisus, Pratylenchus loosi, Pratylenchus minutus, Pratylenchus mulchandi, Pratylenchus musicola, Pratylenchus neglectus, Pratylenchus penetrans, Pratylenchus pratensis, Pratylenchus reniformia, Pratylenchus scribneri, Pratylenchus thornei, Pratylenchus vulnus, and Pratylenchus zeae.

In some embodiments, the nematicidal protein is a Cry protein. Cry proteins are well known to persons of skill in the art. Nematicidal activity of Cry proteins has been described in, e.g., International Publication Nos. WO 2010/027805, WO 2010/027809, WO 2010/027804, WO 2010/027799, WO 2010/027808 and in WO 2007/147029. In some embodiments, the nematicidal protein comprises a Cry14 protein (see, e.g., International Publication No. WO2018119336 and U.S. Provisional Patent Application Ser. No. 62/112,832 (filed Nov. 12, 2020), each of which is incorporated herein by reference in its entirety). In various embodiments, the Cry14 protein is Cry14Aa1 (GENBANK accession number AAA21516) or Cry14Ab1 (also known as Cry14Ab-1; GENBANK accession number KC156652). In some embodiments, the Cry14Ab-1 protein (SEQ ID NO: 1 (amino acid sequence) and SEQ ID NO: 2 (nucleotide sequence)) is as described in International Publication Nos. WO 2018/119361 and WO 2018/119364, as well as variants and fragments thereof. In various embodiments, the nematode-resistant plant expresses a nematicidal Cry14Ab protein having, having about, or having at least or at least about 95, 96, 97, 98, or at least 99% sequence identity to SEQ ID NO. 1.

A number of nucleic acids having nematicidal activity are well known to those of skill in the art, as are molecular biology techniques for creating plants having nematode resistance. In some embodiments, the nucleic acid expressed by the nematode-resistant plant is produced by a sequence as described in, for example, International Publication Nos. WO 2011/82217, WO 2013/078153, WO 2020/243365, WO 2018/005491 and WO 2021/016098.

The molecular biology techniques used to create a nematode-resistant plant include, as non-limiting examples, genome editing to create a nucleic acid resulting in nematicidal activity; expression of an RNA molecule that results in nematicidal activity; and/or expression of a heterologous, or transgenic, nematicidal protein. Other techniques, such as induced mutations, can also be used to create a nematode-resistant plant. In some embodiments, the nematicidal nucleic acid expressed by the nematode-resistant plant is produced by a Cry gene. In some embodiments, the Cry gene is a Cry14 gene. In some embodiments, the Cry14 gene is a Cry14Aa gene or a Cry14Ab gene.

The nematode-resistant plant may express one or more additional nucleic acids introduced by molecular biology techniques in addition to the nucleic acid resulting in nematicidal activity, including but not limited to nucleic acids that provide herbicide tolerance, resistance to coleopteran pests, resistance to lepidopteran pests, resistance to other pests including other insects; and/or disease resistance. Such other nucleic acids may originate, for example, from molecular biology techniques such as but not limited to genome editing activities, expression of RNA, or expression of heterologous proteins. Further, the nematode-resistant plant may express other non-native nucleic acids due to other techniques such as introduction of mutations, introgression of traits via breeding, and/or other techniques well known to those of ordinary skill in the art. The presence of such other nucleic acids and/or traits is not limiting to the methods herein.

In some embodiments, the nematode-resistant plant expresses one or more additional non-native nucleic acids in addition to the nucleic acid providing nematicidal activity. In some embodiments, the nucleic acid providing nematicidal activity is combined with one or more soybean GM events providing tolerance to any one or a combination of glyphosate-based, glufosinate-based, HPPD inhibitor-based, sulfonylurea- or imidazolinone-based, AHAS- or ALS-inhibiting and/or auxin-type (e.g., dicamba, 2,4-D) herbicides, such as Event EE-GM3 (aka FG-072, MST-FG072-3, described in WO2011063411, USDA-APHIS Petition 09-328-01p), Event SYHT0H2 (aka 0H2, SYN-000H2-5, described in WO2012/082548 and 12-215-01p), Event DAS-68416-4 (aka Enlist Soybean, described in WO2011/066384 and WO2011/066360, USDA-APHIS Petition 09-349-01p), Event DAS-44406-6 (aka Enlist E3, DAS-44406-6, described in WO2012/075426 and USDA-APHIS 11-234-Olp), Event MON87708 (dicamba-tolerant event of Roundup Ready 2 Xtend Soybeans, described in WO2011/034704 and USDA-APHIS Petition 10-188-Olp, MON-87708-9), Event MON89788 (aka Genuity Roundup Ready 2 Yield, described in WO2006/130436 and USDA-APHIS Petition 06-178-01p), Event 40-3-2 (aka Roundup Ready, GTS 40-3-2, MON-04032-6, described in USDA-APHIS Petition 93-258-01), Event A2704-12 (aka LL27, ACS-GM005-3, described in WO2006108674 and USDA-APHIS Petition 96-068-Olp), Event 127 (aka BPS-CV127-9, described in WO2010/080829), Event A5547-127 (aka LL55, ACS-GM006-4, described in WO2006108675 and in USDA-APHIS Petition 96-068-01p), event MON87705 (MON-87705-6, Vistive Gold, published PCT patent application WO2010/037016, USDA-APHIS Petition 09-201-01p), soybean Event HB4 (OECD Unique Identifier IND-00410-05, USDA-APHIS Petition 17-223-01p), or event DP305423 (aka DP-305423-1, published PCT patent application WO2008/054747, USDA-APHIS Petition 06-354-01p), or the nucleic acid resulting in nematicidal activity is combined with a combination of the following events: Event MON98788 x MON87708 (aka Roundup Ready 2 Xtend Soybeans, MON-87708-9 x MON-89788-1), Event HOS x Event 40-3-2 (aka Plenish High Oleic Soybeans x Roundup Ready Soybeans), Event EE-GM3 x EE-GM2 (aka FG-072xLL55, described in WO2011063413), Event MON 87701 x MON 89788 (aka Intacta RR2 Pro Soybean, MON-87701-2 x MON-89788-1), DAS-81419-2 x DAS-44406-6 (aka Conkesta™ Enlist E3™ Soybean, DAS-81419-2 x DAS-44406-6), Event DAS-68416-4 x Event MON 89788 (aka Enlist™ RoundUp Ready@ 2 Soybean, DAS-68416-4 X MON-89788-1), Event MON-87769-7 x Event MON-89788-1 (aka Omega-3 X Genuity Roundup Ready 2 Yield Soybeans), Event MON 87705 x Event MON 89788 (aka Vistive Gold, MON-87705-6 x MON-89788-1), or Event MON87769 x Event MON89788 (aka Omega-3 x Genuity Roundup Ready 2 Yield Soybeans, MON-87769-7 x MON-89788-1). In some embodiments, any of the above traits are modified using introduction of mutations, genome editing, or other molecular biology techniques and combined with the nematode-resistant trait, alone or in any combination.

In some embodiments, the nematode-resistant plant is a soybean plant containing the EE-GM4 event as described in International Publication No. WO 2018/119361 or a soybean plant containing the EE-GM5 event (also known as the GMB151 event) as described in International Publication No. WO 2018/119364. The disclosures of International Publication Nos. WO 2018/119361 and WO 2018/119364 are incorporated herein by reference in their entireties.

In some embodiments, the nematode-resistant plant contains one or more native traits that provide resistance to nematodes. Such native traits are well known to those of skill in the art (see, for example, Fosu-Nyarko, J., and M. G. K. Jones. 2015. Application of biotechnology for nematode control in crop plants. Pages 339-376 in: Advances in Botanical Research Vol. 73, Plant Nematode Interactions: A View on Compatible Interrelationships. Chapter 14. C. Escobar and C. Fenoll, eds. Elsevier, Oxford). Native traits may be complementary to the nematode suppressive effect of a nematode-resistant plant and in some embodiments, one or more such native traits are present in the germplasm of the nematode-resistant plant.

Thus, provided herein are methods for killing, suppressing, or controlling a nematode pest population, e.g. Pratylenchus spp. nematode populations, e.g., Pratylenchus brachyurus, Meloidogyne spp., Heterodera spp., e.g., Heterodera glycines, Globodera spp., Rotylenchulus reniformis, Helicotylenchus spp., e.g. Helicotylenchus dihystera, Scutellonema brachyurus, Tubixaba tuxaua, or Aphelencoides besseyi via a nematicidal nucleic acid or protein expressed by a nematode-resistant plant as described herein. In specific embodiments, the nematicidal protein comprises the Cry14 protein set forth in International Publication Nos. WO 2018/119361, WO 2018/119364, or U.S. Provisional Patent Application Ser. No. 62/112,832, as well as variants and fragments thereof.

### Systems

In another aspect, described herein is a system for increasing the efficiency of growing a nematode-susceptible plant, the system comprising: growing a nematode-resistant plant in a locus during a first growing season; and growing the nematode-susceptible plant in the locus during the same or a subsequent growing season. In some embodiments, a second nematode-susceptible plant is grown in the locus subsequent to growing the first nematode-susceptible plant. In some embodiments, the increase in efficiency of growing the nematode-susceptible plant may be indicated or measured by one or more of the following: a. use of the locus for at least one additional growing season per year; b. decreased tillage of the locus; c. decreased treatment of the nematode-susceptible crop seed with nematicides; d. decreased treatment area of the locus with nematicides prior to or during the growing season for the nematode-susceptible plant; e. decreased rate of nematicide applied to the nematode-susceptible plant and/or the locus prior to or during the growing season for the nematode-susceptible plant; f. decreased number of applications of nematicides made to the nematode-susceptible plant and/or the locus during the growing season; increased usability of the locus; increased value of the locus; improved sustainable agricultural practices; and/or g. increased yield from the nematode-susceptible crop.

In another aspect, described herein is a system for increased use of a locus, the system comprising: growing a nematode-resistant plant in a locus in a first growing season; and growing a first nematode-susceptible plant in the locus in the same or a subsequent growing season. In some embodiments, the nematode-resistant plant and the first nematode-susceptible plant are grown in the same or consecutive growing seasons. In other embodiments, the nematode-resistant plant and the first nematode-susceptible plant are grown in non-consecutive growing seasons. In some embodiments, the locus is not fallowed over the course of a subsequent growing season. In some embodiments, no cover crop is planted over the course of a subsequent growing season. In some embodiments, the locus is not tilled.

In another aspect, described herein is a system for increased use of a locus, such as but not limited to a field or a plot, the system comprising: growing a nematode-resistant plant in a locus in a first growing season; growing a first nematode-susceptible plant in the locus in the same or a subsequent growing season; and growing a second nematode-susceptible crop in the locus subsequent to growing the first nematode-susceptible crop in the locus. In some embodiments, the nematode-resistant plant, the first nematode-susceptible plant, and the second nematode-susceptible plant are grown in consecutive growing seasons. In other embodiments, the nematode-resistant plant, the first nematode-susceptible plant, and the second nematode-susceptible plant are grown in non-consecutive growing seasons, or only two of the three are grown in consecutive or non-consecutive growing seasons. In some embodiments, the locus is not fallowed over the course of a subsequent growing season. In some embodiments, no cover crop is planted over the course of a subsequent growing season. In some embodiments, the locus is not tilled.

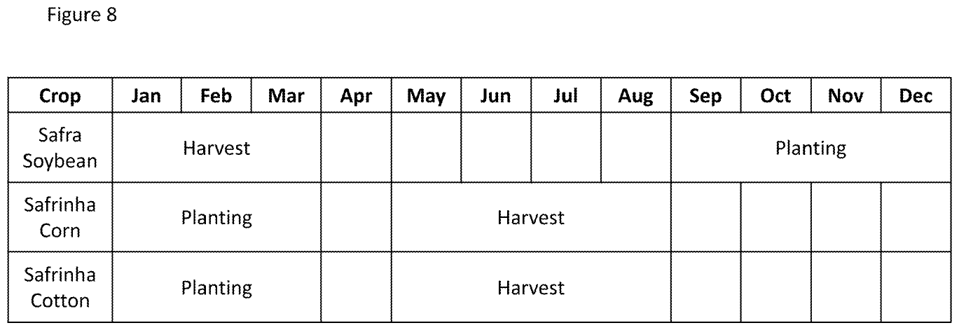

The term “crop rotation system” or “cropping system” as used herein refers to the practice of following a crop rotation cycle in which one agricultural crop planted in a locus, for example a field, is followed with one or more successive crops within a period of time. The period of time encompassed by a single crop rotation cycle does not necessarily correspond with a calendar year, nor is it necessarily a twelve-month cycle. In some instances, a single crop rotation cycle may span a time period of two to three years or even longer. In some embodiments, sugarcane is on about a 5-year cycle (e.g., 4-5 years of sugarcane, followed by one season of non-sugarcane). The precise timing of the plantings, the period of time of a cycle, and the crops to be planted are dictated by the environment and are specific to a location. Crop rotation systems are used for a number of reasons, such as considerations of each crop's nutrient demands, reduction of disease and/or pest pressure, and business diversification. As an example, a typical Brazilian soybean cropping system produces two crops that are planted in succession in a calendar year. The primary growing season, called the “safra”, begins with planting a first crop, such as soybeans, in September-December. The harvest of a safra season crop takes place between January and March. Immediately after harvest of the safra season crop the second season crop, termed “safrinha”, is planted. The safrinha crops are then harvested in May-August. See FIG. 8. In the United States, the crop rotation cycle varies depending on the environment and the goals of the farmer, and is not necessarily tied to a calendar year. In one example, the primary growing season begins with planting a primary crop in February-May, with harvest occurring in August-October. A second crop is then planted in September-November, or planted the following year in February-May. Other locations or geographies support growth of crops during a crop rotation cycle of three growing seasons, and still others support growth of crops during a crop rotation cycle of four growing seasons or more.

In one aspect, described herein is a method of suppressing a nematode population in a locus, comprising growing a nematode-resistant plant in a locus, wherein growing the nematode-resistant plant suppresses a nematode population in the locus or maintains the suppression of the nematode population in the locus for a period of time during and/or beyond the growing of the nematode-resistant plant. In some embodiments, the nematode-resistant plant is grown during the safra season. In some embodiments, the methods further comprise growing a secondary plant in the locus during the safrinha season immediately following the safra season. In some embodiments, the secondary plant is a nematode-susceptible plant. In some embodiments, both the nematode-resistant plant and the secondary plant are intrinsic value crop plants. In some embodiments, both the nematode-resistant plant and the secondary plant are cash crop plants. In some embodiments, the nematode-resistant plant is a soybean plant and the secondary plant is selected from the group consisting of corn, cotton, sorghum, wheat and sugarcane.

In some aspects, described herein is a method for improving a crop rotation system, the method comprising: growing a nematode-resistant plant in a locus during a first growing season; and growing the nematode-susceptible plant in the locus during the same or a subsequent growing season, wherein growing the nematode-resistant plant in the locus during the first growing season results in a suppression of the nematode population in the locus that enables or improves the growth of the nematode-susceptible plant in the same or subsequent growing season. In some embodiments, a second nematode-susceptible plant is grown in the locus subsequent to growing the first nematode-susceptible plant.

In some embodiments, the improved crop rotation system may be indicated or measured by one or more of the following: a. use of the locus for at least one additional growing season per year; b. decreased tillage of the locus; c. decreased treatment of the nematode-susceptible crop seed with nematicides; d. decreased treatment area of the locus with nematicides prior to or during the growing season for the nematode-susceptible plant; e. decreased rate of nematicide applied to the nematode-susceptible plant and/or the locus prior to or during the growing season for the nematode-susceptible plant; f. decreased number of applications of nematicides made to the nematode-susceptible plant and/or the locus during the growing season; increased usability of the locus; increased value of the locus; improved sustainable agricultural practices; and/or g. increased yield from the nematode-susceptible crop.

It is to be understood that this invention is not limited to the particular methodology, protocols, plant species or genera, constructs, and reagents described as such. It is also to be understood that the terminology used herein is for the purpose of describing particular embodiments only, and is not intended to limit the scope of the present invention, which will be limited only by the appended claims. It must be noted that as used herein and in the appended claims, the singular forms “a,” “and,” and “the” include plural reference unless the context clearly dictates otherwise. Thus, for example, reference to “a vector” is a reference to one or more vectors and includes equivalents thereof known to those ordinarily skilled in the art, and so forth.

### Marketing

As used herein, “marketing” is any communication by or on behalf of an entity for the purpose of alerting the public/potential customers to commercial products and to encourage acceptance and/or uses thereof. Print marketing/materials may include but is not limited to brochures, pamphlets, flyers, catalogs, business cards, signs, posters, billboards, trade literature, seed bags, seed bag tags, labels, or other publicity. Other types of marketing/materials may include but is not limited to any type of digital information, including information on websites, emails, texts, any company-sponsored communication or event to promote/disclose a product, or any social media platform. Marketing also includes any rebates/incentives to a farmer directed to the purchase of one or more products, the use of which in the course of a crop rotation cycle is/are improved by the use of a nematode-resistant plant according to the methods provided herein.

In some aspects, the disclosure also provides methods for marketing a crop rotation system comprising promoting use of a nematode-resistant plant in a locus during a first growing season; and promoting use of a nematode-susceptible plant in the locus during the same or a subsequent growing season, wherein growing the nematode-resistant plant in the locus during the first growing season results in a suppression of the nematode population in the locus that enables or improves the growth of the nematode-susceptible plant in the subsequent growing season. In further aspects, methods marketing a crop rotation system for suppressing a nematode population in a locus are provided comprising promoting use of a nematode-resistant plant in the locus during a first growing season; and promoting use of a nematode-susceptible plant in the locus during the same or a subsequent growing season, wherein growing the nematode-resistant plant in the locus during the first growing season results in a suppression of the nematode population in the locus that enables or improves the growth of the nematode-susceptible plant in the subsequent growing season.

In further aspects, the disclosure provides marketing materials directed to a system of growing a nematode-resistant plant in a locus during a first growing season in coordination with or followed by growing a nematode-susceptible plant in the locus during a subsequent growing season, wherein growing the nematode-resistant plant in the locus during the first growing season results in a suppression of the nematode population in the locus that enables or improves the growth of the nematode-susceptible plant in the subsequent growing season. In some embodiments, the marketing materials are directed to promoting a system of growing a nematode-resistant plant in a locus during a first growing season in coordination with or followed by growing a nematode-susceptible plant in the locus during a subsequent growing season, wherein growing the nematode-resistant plant in the locus during the first growing season results in a suppression of the nematode population in the locus that enables or improves the growth of the nematode-susceptible plant in the subsequent growing season.

In some aspects, the disclosure provides a method for improving the marketing of a crop rotation system to reduce nematode populations, reduce injury to a crop from nematodes, and/or increase crop yields via suppression of nematode populations, the method comprising characteristics such as but not limited to one or more of a rebate program, marketing statements, a connected offer, or an integrated solution, or any sales offer directed to the purchase of one or more products, the use of which in the course of a crop rotation cycle is/are improved by the use of a nematode-resistant plant according to the methods provided herein.

In further aspects, the disclosure provides a kit comprising marketing materials as described herein (e.g., related to marketing a crop rotation system) and/or a nematode-resistant soybean plant, or cells, parts, seeds or progeny thereof, each as described herein. In some embodiments, the kit further comprises a secondary plant, or cells, parts, seeds or progeny thereof. In some embodiments, the kit further comprises any product useful in growing the plants, cells, parts, seeds, or progeny thereof, including but not limited to herbicides, fungicides, insecticides, nematicides, biologics, fertilizers, seed treatments, inoculants, decision making or remote sensing tools, crop scouting services, crop diagnostic tools and/or services. In any of the aspects or embodiments of the disclosure, a kit is any package or material that includes marketing of a system or a method for suppressing or maintaining a nematode population in a locus, such that the resulting nematode population in the locus is about 250 nematodes per gram (g) of root or less. In various embodiments, a kit is any package or material that includes marketing of a system or a method for suppressing or maintaining a nematode population in a locus, wherein the resulting nematode population in the locus is, is about, or is less than about 250, 200, 150, 100, 50, 20, or 10 nematodes per gram (g) of root.

As described herein, the products, systems, and methods of the disclosure also provide improved value and sustainability by enabling improved agricultural practices (e.g., through carbon credit opportunities). For example, sustainability opportunities created by embodiments of the disclosure may include but are not limited to (i) improving a locus so that it requires less tillage; (ii) eliminating the need to grow cover crops or leave a locus fallow during at least one growing season per year; and/or (ii) enabling growth of plants with healthier and larger root systems that can capture more carbon than plants with smaller root systems or roots damaged by nematodes. A further related advantage provided by the disclosure relates to improved nitrogen fixation. Nematodes reduce nitrogen fixation in soybeans, thereby increasing the chemical nitrogen needs of a crop (e.g., a corn crop) planted the following season. In any of the aspects or embodiments of the disclosure, the nematode-resistant plants as described herein suppress the nematode population and enable improved or full nitrogen fixation in the soybeans, which then decreases the chemical nitrogen needs of the corn crop the following season. In addition, chemical nitrogen causes environmental issues and is of concern (see, e.g., Crews et al., Agriculture, Ecosystems and Environment 102 (2004) 279-297).

### Embodiments

1. A method of suppressing a nematode population in a locus, the method comprising:
growing a nematode-resistant plant in a locus, wherein growing the nematode-resistant plant suppresses a nematode population in the locus or maintains the suppression of the nematode population in the locus for a period of time during and/or beyond the growing of the nematode-resistant plant.
2. The method of embodiment 1, wherein the period of time extends to one or more growing seasons subsequent to the growing season in which the nematode-resistant plant was grown.
3. The method of embodiment 1 or 2, further comprising growing a secondary plant in the locus subsequent to growing the nematode-resistant plant.
4. The method of embodiment 1 or 2, further comprising growing a secondary plant in the locus prior to growing the nematode-resistant plant.
5. The method of embodiment 1 or 2, further comprising growing the nematode-resistant plant at the same time as a secondary plant in the locus.
6. The method of any one of embodiments 3-5, wherein the secondary plant is a nematode-susceptible plant.
7. The method of any one of embodiments 1-6, wherein the nematode population is suppressed or maintained at or below the limit of detection.
8. The method of any one of embodiments 3-6, wherein the secondary plant is Brachiaria and the suppressing of the nematode population is achieved when the number of nematodes is, is about, or is less than about 60 nematodes per gram of root.
9. The method of any one of embodiments 3-6, wherein the secondary plant is corn and the suppressing of the nematode population is achieved when the number of nematodes is, is about, or is less than about 300 nematodes per gram of root.
10. The method of any one of embodiments 3-6, wherein the secondary plant is cotton and the suppressing of the nematode population is achieved when the number of nematodes is, is about, or is less than about 60 nematodes per gram of root.
11. The method of any one of embodiments 3-6, wherein the secondary plant is sorghum and the suppressing of the nematode population is achieved when the number of nematodes is, is about, or is less than about 250 nematodes per gram of root.
12. The method of any one of embodiments 3-11, wherein the suppressing of the nematode population in the locus in which the secondary plant is grown is achieved when there is, is about, or is at least about a 5% reduction in the number of nematodes per gram of root relative to the number of nematodes per gram of root in a comparable locus to the locus in which the secondary plant is grown.
13. A method of protecting a plant from nematode injury or damage comprising:
growing a nematode-resistant plant in a locus at least one growing season before planting a nematode-susceptible plant; and
growing a nematode-susceptible plant in the locus at least one growing season subsequent to growing the nematode-resistant plant.
14. The method of embodiment 13, wherein the protection of the nematode-susceptible plant from nematode damage comprises an increase in yield from and/or money made from the sale of harvested nematode-susceptible plant material.
15. A method of increasing yield of a nematode-susceptible plant from comprising:
growing a nematode-resistant plant in a locus at least one growing season before planting the nematode-susceptible plant; and
growing a nematode-susceptible plant in the locus at least one growing season subsequent to growing the nematode-resistant plant.
16. The embodiment of embodiment 15, wherein the increased yield of the nematode-susceptible plant is compared to the yield of the nematode-susceptible plant grown in the same or a comparable locus that has not previously grown the nematode-resistant crop, or the increased the yield of the nematode-susceptible plant is compared to the yield of the nematode-susceptible plant grown in the same locus prior to growing the nematode-resistant plant or a comparable locus.
17. A method of nematode management for a locus, the method comprising:
growing a nematode-resistant plant in a locus; and
growing a nematode-susceptible plant in the locus at the same time or at a time subsequent to growing the nematode-resistant plant.
18. The method of embodiments 6-17, wherein the nematode-susceptible plant is a perennial plant or an annual plant.
19. The method of any one of embodiments 6-18, wherein the nematode-susceptible plant is a soybean plant, a corn plant, a cotton plant, a canola plant, a sugarcane plant, a potato plant, a wheat plant, a vegetable plant, a rice plant, an alfalfa plant, a barley plant, a sorghum plant, an oat plant, a rye plant, a cassava plant, a sweet potato plant, a sunflower plant, a fruit plant, an orchard plant (such as a fruit or nut tree), an ornamental plant, or a grape vine.
20. The method of any one of embodiments 1-19, wherein the nematode-resistant plant expresses a Cry protein, for example and without limitation a nematicidal Cry protein.
21. The method of any one of embodiments 1-20, wherein the nematode-resistant plant is a soybean plant, a corn plant, a cotton plant, a canola plant, a sugarcane plant, a sugar beet plant, a potato plant, a wheat plant, a rice plant, an alfalfa plant, a barley plant, a sorghum plant, an oat plant, a rye plant, a cassava plant, a sweet potato plant, a sunflower plant, a vegetable plant, a fruit plant, an ornamental plant, an orchard plant (such as a fruit or nut tree), or a grape vine.
22. The method of any one of embodiments 1-21, wherein the nematode is a nematode species selected from Pratylenchus spp. nematode populations, e.g., Pratylenchus brachyurus, Meloidogyne spp., Heterodera spp., e.g. Heterodera glycines, Globodera spp., Rotylenchulus reniformis, Helicotylenchus spp., e.g. Helicotylenchus dihystera, Scutellonema brachyurus, Tubixaba tuxaua, or Aphelencoides besseyi.
23. The method of embodiment 22, wherein the Pratylenchus spp is Pratylenchus brachyurus.
24. A method of nematode management for a locus, the method comprising:
growing a nematode-resistant plant in a locus at the same time as a nematode-susceptible plant.
25. A method of nematode management for a locus, the method comprising: planting a nematode-susceptible plant in a locus; and growing a nematode-resistant plant in the locus at a time following the planting of the nematode-susceptible plant.
26. A locus having a suppressed nematode population density, wherein the suppressed nematode population density is achieved by the method of any one of embodiments 1-25.
27. The locus of embodiment 26 that provides one or more of the following benefits: the locus does not does not need to lie fallow one growing season per year or per crop rotation cycle; b. the locus does not need to be tilled one growing season per crop rotation cycle; and c. the locus does not need to be planted with a cover crop.
28. The locus of embodiment 26 or embodiment 27, wherein the suppressed nematode population density is, is about, or is less than about 250, 200, 150, 100, 50, 20, or 10 nematodes per gram (g) of root.
29. The locus of embodiment 26 that does not does not need to lie fallow one growing season per crop rotation cycle.
30. A nematode-resistant plant grown in the locus of embodiment 26.
31. Plant material harvested from the plant of embodiment 30.
32. Seeds produced by the plant of embodiment 31.
33. A system for increased use of a locus, the system comprising:
growing a nematode-resistant plant in a locus in a first growing season; and
growing a nematode-susceptible plant in the locus in a subsequent growing season.
34. The system of embodiment 33, wherein the locus is not fallowed during a subsequent growing season.
35. The system of embodiment 33, wherein no cover plant or cover crop is grown during the subsequent growing season.
36. The system of embodiment 33, wherein the nematode-resistant plant and the nematode-susceptible plant are grown in consecutive growing seasons.
37. The system of any one of embodiments 33-36, wherein each of the nematode-resistant plant and the nematode-susceptible plant is an intrinsic value crop plant.
38. A method for improving a crop rotation system, the method comprising:
growing a nematode-resistant plant in a locus during a first growing season; and
growing a nematode-susceptible plant in the locus during a subsequent growing season, wherein growing the nematode-resistant plant in the locus during the first growing season results in a suppression of the nematode population in the locus that enables or improves the growth of the nematode-susceptible plant in the subsequent growing season.
39. The method of embodiment 38, wherein the improved crop rotation system may further include one or more of the following:
a. use of the locus for at least one additional growing season per year;
b. decreased tillage of the locus;
c. decreased treatment of the nematode-susceptible crop seed with nematicides;
d. decreased treatment area of the locus with nematicides prior to or during the growing season for the nematode-susceptible plant;
e. decreased rate of nematicide applied to the nematode-susceptible plant and/or the locus prior to or during the growing season for the nematode-susceptible plant;
f. decreased number of applications of nematicides made to the nematode-susceptible plant and/or the locus during the growing season;
g. increased usability of the locus;
h. increased value of the locus;
i. improved sustainable agricultural practices; and/or
j. increased yield from the nematode-susceptible crop.
40. A method of nematode management for a locus, the method comprising:
growing a nematode-resistant plant in a locus during a growing season; and
growing a perennial plant in the locus before, during, and/or after the growing season, wherein growing the nematode-resistant plant in the locus results in a suppression of the nematode population in the locus that enables or improves the growth of the perennial plant.
41. The method of embodiment 40, wherein the perennial plant is a nematode-susceptible plant.
42. The method of embodiment 40, wherein the perennial plant is a vegetable plant, a fruit plant, an orchard plant, an ornamental plant, or a grape vine.
43. A method of nematode management for a locus, the method comprising:
growing a nematode-resistant plant in a locus in a first growing season, wherein growing the nematode-resistant plant in the first growing season suppresses a nematode population in the locus or maintains the suppression of the nematode population in the locus;
growing a nematode-susceptible plant in the locus in the same or a subsequent growing season; and
achieving improved health and/or yield of the nematode-susceptible plant compared to the health and/or yield expected if the nematode population was not suppressed.
44. The method of embodiment 43, wherein the improved health of the nematode-susceptible plant comprises one or more of the following: improved root development (e.g., improved root or root hair growth); improved yield; faster emergence; improved plant stress management including increased stress tolerance and/or improved recovery from stress; increased mechanical strength; improved drought resistance; reduced fungal, bacterial and/or viral disease infection; or any combination thereof.
45. Any of embodiments 1-44 above, wherein the nematode-resistant plant expresses a nematicidal Cry14Ab protein having at least 95, 96, 97, 98, or at least 99% sequence identity to SEQ ID NO. 1.
46. Any of embodiments 1-44, wherein the nematode resistant plant comprises elite event EE-GM5.
47. Any of embodiments 1-44, wherein the nematode resistant plant comprises elite event EE-GM4.
48. Any of embodiments 1-44, wherein the nematode-resistant plant expresses Bacillus thuringiensis toxin Cry14Ab-1 or a functional truncation or variant thereof.
49. A method of nematode management for a locus, the method comprising:
growing a nematode-resistant plant in a locus, wherein the nematode resistant plant is grown during the safra season in a South American country such as Brazil or Paraguay; and
growing a nematode-susceptible plant in the locus subsequent to growing the nematode-resistant plant, wherein the nematode-susceptible plant is grown during the safrinha season in a South American country such as Brazil or Paraguay.
50. The method of embodiment 49, wherein the nematode-resistant plant expresses Bacillus thuringiensis toxin Cry14Ab-1 or a functional truncation or variant thereof, and the nematode-susceptible plant is selected from the group consisting of corn, cotton, sorghum, wheat, and sugarcane.

The following examples are offered by way of illustration and not by way of limitation.

### EXAMPLES

### Example 1—Generation of Nematode-Resistant Plants

Soybean lines containing the GMB151 transgenic event (i.e., EE-GM5, as described in International Publication No. WO 2018/119364) were created from BC2:F2 selected plants. The soybean lines differed in their zygosity for the GMB151 event. One line homozygous for the GMB151 event was created, while a second line nullizygous for the GMB151 event was also selected. These two lines shared ˜87.5% genetic identity with the common backcross parent soybean line. Therefore, the lines were agronomically similar except for the presence of the GMB151 event.

### Example 2—Control of the Root Lesion Nematode Pratylenchus brachyurus (Godfrey) by GMB151 Transgenic Soybean Expressing the Bacillus thuringiensis Toxin Cry14Ab-1

The ability of Cry14Ab-1 to control P. brachyurus in the field was investigated. Specifically, it was contemplated that the Cry14Ab-1-expressing event GMB151 could reduce P. brachyurus reproduction in field grown soybean and that this reduction in P. brachyurus reproduction would result in significant yield protection.

### Materials and Methods:

Field trials were conducted in the states of Parani, Goias and Mato Grosso, Brazil, during the 2018/19 soybean growing season. Field trial locations are described below in Table 1:

TABLE 1
Pratylenchus brachyurus research trial locations:

| | Expected | | |
|---|---|---|---|
| | Pratylenchus | Population | Seed |
| Location | Pressure^{a} | Density^{b} | Treatments^{c} |
| Ibiporã, Paraná | Low | ND^{d} | No |
| Lupinópolis, Paraná | High | 47 | Yes |
| Rio Verde, Goiás | High | 462 | Yes |
| Sinop1, Mato Grosso | High | 976 | Yes |
| Sinop2, Mato Grosso | Low | 286 | No |
| Trinidade, Goiás | Low | 1 | No |

^{a}Pratylenchus brachyurus pressure as determined by previous year soil samples, crop injury, and farm manager input. The Sinop2 site was expected to have low Pratylenchus pressure based on the farm manager's input, however pressure was relatively high.
^{b}Actual Pratylenchus brachyurus population densities at each research site. Population densities are reported as the average population recovered from the Nullizygous treatment 90 days after planting. Population densities are reported as P. brachyurus per g of root tissue.
^{c}Inclusion of seed treatment sub-plots in the trial. Seed treatments were only included in research trials at sites where high P. brachyurus pressure was expected.
^{d}Not detected.

The GMB151 event was introgressed into a soybean maturity group IX background, which is adapted for production in Brazil. Introgression was accomplished by utilizing the maturity group IX line as the recurrent parent. At the BC2:F2 generation plants homozygous and nullizygous for the GMB151 event were selected to create two soybean lines that differed in the presence or absence of the GMB151 event, but otherwise were approximately 87.5% genetically related to the recurrent parent.

Soybean trials were established at six research sites across a gradient of population densities of P. brachyurus as indicated in Table 1. Three research sites contained P. brachyurus at population densities expected to reduce soybean yields, while three sites were not anticipated to have P. brachyurus at high enough population densities to significantly reduce soybean yield. Expectations of P. brachyurus pest pressure were made based on 2017/18 soybean crop samples or communications with farm managers.

Differences in experimental design and trial treatments differed among the anticipated high- and low-pressure sites. Anticipated low-pressure trial sites were conducted as randomized complete block design trials with five replications. Each field plot consisted of four 5 m-long soybean rows spaced 0.5 m apart. Two treatments were included in each trial, a GMB151 homozygous line (Homozygous) and a GMB151 nullizygous line (Nullizygous).

At the three high-pressure sites, two changes were made in the trials compared to the low-pressure sites. First, the GMB151 event was evaluated in combination with nematicidal seed treatments. Three seed treatments were included in the trial. The treatment factor of seed treatment was fully crossed with the treatment factor of zygosity (i.e. Homozygous or Nullizygous) to create six treatments. The six treatments were arranged in a split-plot design with zygosity as the whole-plot treatment and seed treatment as the sub-plot treatment. Sub-plots remained the same size as other sites, four 5 m-long soybean rows spaced 76.2 cm apart. The second change made to trials at the high-pressure sites was to reduce the number of replications from five to three. The number of replications was reduced due to the inclusion of seed treatment sub-plots and limitations in space available for field trials.

### Data Collection:

The efficacy of GMB151 against P. brachyurus was evaluated by measuring the population density of P. brachyurus within soybean root systems at 90 days after planting (dap). The 90 dap time point was selected as it approximated the sampling time of previous P. brachyurus studies (Lima et al. 2015). At 90 dap the soybean root systems of ten plants per plot were removed from the field. Five root systems were sampled from the first and fourth row of each plot. These rows were selected as they were not harvested and therefore P. brachyurus sampling would not affect yield estimates taken at the end of the season. Root samples were taken to the laboratory where they were weighed and then homogenized in a blender. Nematodes were then extracted from the root pieces using the methods of Jenkins (1964). Extracted nematodes were then suspended in water and the number of P. brachyurus in a 1 ml subsample was enumerated under a microscope. The number of P. brachyurus per g of root was calculated for each sample.

Soybean yield was estimated for each plot at physiological maturity. The middle two rows of each four-row plot were harvested. Grain weight and moisture were measured for each plot and yield was standardized to 13% moisture.

### Data Analysis:

Pratylenchus brachyurus population density data was analyzed separately for the three high-pressure locations and the three low-pressure locations. The number of P. brachyurus per gram (g) of root was natural log transformed prior to analysis to reduce heteroscadacity. Yield data for each trial were analyzed separately due to the wide range of P. brachyurus population densities present across the research trial locations, which would affect the amount of injury caused by P. brachyurus. Yield data was analyzed as bushels per acre.

Data were analyzed to estimate the effect GMB151 zygosity had on P. brachyurus population densities and soybean yield. Data were fit to a mixed effects model ANOVA and the difference between the Homozygous and Nullizygous lines was estimated with a 95% confidence interval. Trials that did not include seed treatment sub-plots utilized a model that included replication as a random effect and zygosity as a fixed effect. The interaction of replication and zygosity was considered the error term. Trials that included seed treatment sub-plots included zygosity, seed treatment, and the interaction of zygosity by seed treatment as fixed effects. Replication and all other interactions among factors were considered random effects. The interaction of replication and zygosity was considered the whole plot error term, while the interaction of replication, zygosity, and seed treatment was considered the sub-plot error term. The effect of seed treatments on P. brachyurus, soybean yield, and their interaction with the GMB151 trait is beyond the scope of the study reported here. Therefore, data were only summarized by zygosity across seed treatments.

### Results:

Pratylenchus brachyurus population densities in two high-pressure (Rio Verde and Sinop1) and one low-pressure location (Sinop2) were above those observed to cause aboveground disease symptoms by Lima et al. (2015). The high-pressure Lupinópolis location averaged P. brachyurus population densities that were within the range Lima et al. (2015) observed outside symptomatic field areas and therefore would be expected to result in less yield reduction, if any. The final two low-pressure locations (Ibiporá and Trinidade) had P. brachyurus populations at the limit of detection and therefore should not affect soybean yield.

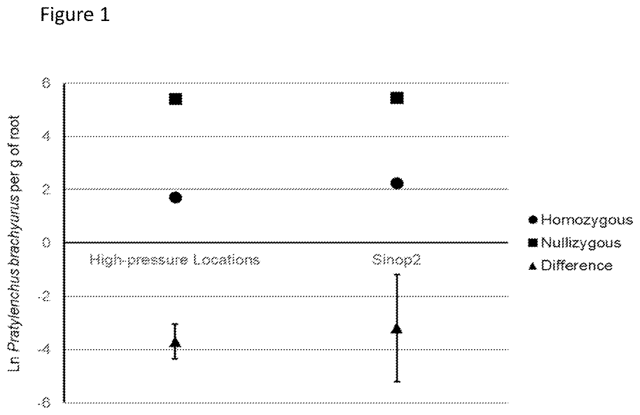

The Homozygous soybean line reduced P. brachyurus population densities by 96% across the three high-pressure locations (P<0.0001). The Homozygous line also significantly reduced P. brachyurus populations by 90% at the anticipated low-pressure location, Sinop2 (P <0.01). At the individual trial locations, average P. brachyurus reductions ranged from 90%-97% (FIG. 1).

Soybean yield was negatively affected at both the Lupinópolis and Ibiporá sites by a poor maturity group fit to the region. The poor fit resulted in low absolute yields across all trial treatments. Treatment yields ranged from 12.4 bu/acre to 16.9 bu/acre at these two trial sites. Treatment yields at the other four locations ranged from 28.0 to 57.1 bu/acre.

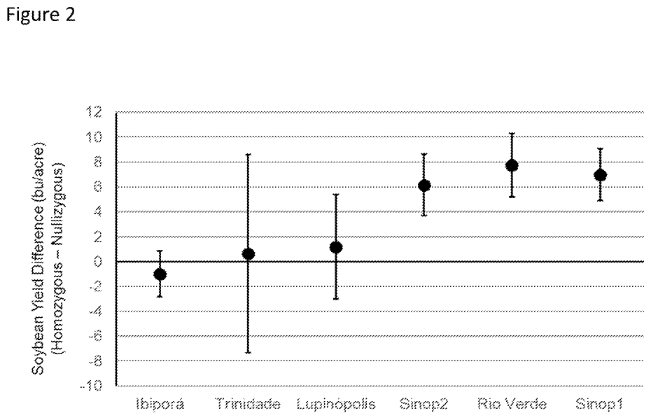

The yield difference among Homozygous and Nullizygous lines was dependent upon the population density of P. brachyurus at the trial location (FIG. 2). There was not a significant yield difference between the two soybean lines at any of the three locations with the lowest P. brachyurus population densities. The three sites with the highest P. brachyurus population densities each had a significant yield difference between the Homozygous and Nullizygous lines. The average yield improvement of the Homozygous line compared to the Nullizygous line at each location was 6.2-7.8 bu/acre or 14-25%.

### DISCUSSION

The six research sites selected ranged in Pratylenchus brachyurus population densities from below the limit of detection to nearly 1,000 nematodes per gram of root tissue. The six sites allowed us to evaluate the efficacy of the GMB151 transgenic soybean event against P. brachyurus and its ability to protect soybean yield. The GMB151 event was highly efficacious against P. brachyurus. Three months into the growing season, P. brachyurus populations were >90% lower on average in the soybean line homozygous for the GMB151 event as compared to the nullizygous line. This level of control far exceeds that provided by current management practices, including crop rotation, fallowing, and chemical control (Lima et al. 2015, Ribeiro et al. 2014, Rodrigues et al. 2014).

The benefit of the GMB151 event for soybean yield protection was dependent upon the population density of P. brachyurus. Under low P. brachyurus pressure, soybean yield was not affected by the GMB151 trait. This result indicates in the absence of P. brachyurus, GMB151 does not have any negative effects on soybean yield. In each of the three trial locations with the highest P. brachyurus population densities, soybean yield was significantly greater in the soybean line homozygous for the GMB151 trait. Soybean yield improvements ranged from 14-25% at these sites. Our results are therefore in agreement with previous studies that estimated the yield injury of P. brachyurus at between 10-30% (Dias et al. 2007, Franchini et al. 2007).

The results of the field trials reported here indicate that the Cry14Ab1-expressing soybean trait GMB151 provided significant efficacy against P. brachyurus, resulting in improved soybean yields. These results were obtained under field production conditions with P. brachyurus population densities within the range reported for commercial production fields in Brazil (Franchini et al. 2007, Lima et al. 2015). Therefore, the GMB151 trait represents a potentially unique new management tool for controlling an economically important crop pest with few viable management options today.

### References for Example 2

Barbosa, B. F. F., J. M. dos Santos, J. C. Barbosa, P. L. M. Soares, A. R. Ruas, and R. B. de Carvalho. 2013. Aggressiveness of Pratylenchus brachyurus to the sugarcane, compared with key nematode P. zeae. Nematropica 43: 119-130.
Dias, W. P., N. R. Ribeiro, I. O. N. Lopes, A. Garcia, G. E. S. Carneiro, and J. F. V. Silva. 2007. Manejo de nematoides na cultura da soja. In: Congresso Brasileiro de Nematologia; 12 August; Goiana, Brazil. Goiana: Sociedade Brasileira de Nematologia. p 26-30.
Franchini, J. C., O. F. Saraiva, H. Debiase, and S. L. Goncalves. 2007. Contribuição de sistema de manejo do solo para produção sustentável da soja. Circular Técnica 46:1-4.
Inomoto, M. M., A. M. C. Goulart, A. C. Z. Machado, and A. R. Monteiro. 2001. Effect of population densities of Pratylenchus brachyurus on the growth of cotton plants. Fitopatologia Brasileira 26: 192-196.
Jenkins, W. R. 1964. A rapid centrifugal-flotation technique for separating nematodes from soil. Plant Disease Reporter 48: 692.
Lima, F. S. D. O., G. R. D. Santos, S. R. Nogueira, P. R. R. D. Santos, and V. R. Correa. 2015. Population dynamics of the root lesion nematode, Pratylenchus brachyurus, in soybean fields in Tocantins State and its effect to soybean yield. Nematropica 45: 170-177.
Lima, F. S. O., V. R. Correa, S. R. Nogueira, and P. R. R. Santos. 2017. Nematodes affecting soybean and sustainable practices for their management. Intech doi: 10.5772/67030.
Ribeiro, N. R., W. P. Dias, and J. M. Santos. 2010. Distribuição de fitonematoides em regiões produtoras de soja no estado do Mato Grosso. Boletim de Pesquisa de Soja. Fundação MT, Rondonópolis, MT, 289-296.
Ribeiro, L. M, H. D. Campos, C. R. Dias-Arieira, D. L. Neves, and G. C. Ribeiro. 2014. Effect of soybean seed treatment on the population dynamics of Pratylenchus brachyurus under water stress conditions. Bioscience Journal 30: 616-622.
Rios, A. D. F., M. R. Rocha, A. S. Machado, K. A. G. B. Avila, R. A. Teixeira, L. C. Santos, and L. R. S. Rabelo. 2016. Host suitability of soybean and corn genotypes to the lesion caused by nematode under natural infestation conditions. CiênciaRural. 46: 580-584.
Rodrigues, D. B., C. R. Dias-Arieira, M. V. V. Vedoveto, M. Roldi, H. F. D. Molin, and V. H. F. Abe. 2014. Crop rotation for Pratylenchus brachyurus control in soybean. Nematropica 44: 146-151.
Wei, J., K. Hale, L. Carta, E. Platzer, C. Wong, S. Fang, and F. V. Aroian. 2003. Bacillus thuringiensis crystal proteins that target nematodes. Proceedings of the National Academy of Sciences 100: 2760-2765.

### Example 3-GMB151 Transgenic Soybean Expressing the Bacillus thuringiensis Toxin Cry14Ab-1 Provides Nematicidal Protection in Safrinha (“Second Harvest”) Crops

The Brazilian soybean cropping system can produce two crops in a calendar year. The primary growing season, called the “safra”, begins with planting soybeans in September-December. The harvest of safra season soybean crop takes place between January and March. Immediately after soybean harvest the second season crop, termed “safrinha”, is planted. The safrinha crops are then harvested in May-August. In the soybean cropping system, the most common safrinha crops in Brazil are corn and cotton. In Brazil, soybean, corn, and cotton are all susceptible to the plant-parasitic nematode Pratylenchus brachyurus. Thus, determining whether planting GMB151 soybean in the safra season could provide nematode suppression to subsequent nematode susceptible safrinha season crops was examined.

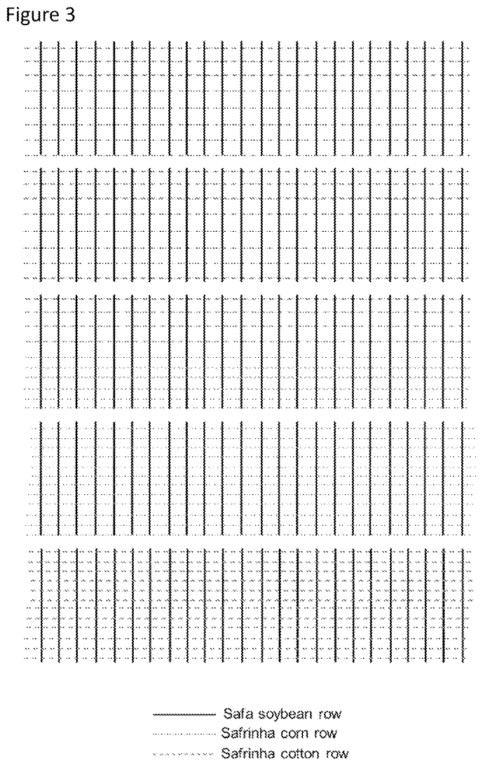

Two soybean lines, one homozygous and one null for the GMB151 trait, were planted in production fields infested with P. brachyurus. Three P. brachyurus infested fields were utilized in 2018 and 2019 for the study. Soybean field trials consisted of three (2018) or five (2019) replications arranged in a randomized complete block design with individual plots measuring 6 meters in length by 12 soybean rows wide. Immediately after soybean trial harvest in April/May 2019 and 2020 a safrinha crop was planted on top of the same ground. In 2019, signal grass was planted as the safrinha crop. Signal grass plots were planted parallel to harvested soybean rows at each location. Three replications of signal grass were planted on top of the soybean field trial ground. Plots were therefore 6 meters in length by 12 rows wide. In April 2020, corn and cotton were planted as the safrinha crops following soybean. Three replications of corn plots and three replications of cotton plots were planted on top of the soybean trial ground. Corn and cotton rows were planted perpendicular to the harvested soybean rows. Cotton plots were planted on top of replications one, two, and the first three m of replication three. Corn plots were planted on top of replications five, four and the last three m of replication three. Cotton and corn plots were therefore 9 m in length, with two replications being 8 rows wide, and the third replication of each crop being 4 rows wide (FIG. 3).

Data were collected to determine if protection from P. brachyurus would be provided to safrinha crops planted following the GMB151 soybean. Protection to safrinha crops was assessed by measuring P. brachyurus population densities and crop yield. Pratylenchus brachyurus population densities were measured as the number of nematodes per g of root tissue. Population densities of P. brachyurus were measured at 30 days after planting (dap), 60 dap and 90 dap in the safrinha crops. The root system of ten plants per plot were collected for each P. brachyurus measurement. Five roots were collected from the first row and last row of each plot. The middle rows were not sampled as root samples are destructive and would interfere with the collection of yield data from the middle rows of each plot. Grain yield was collected from the middle rows of the cotton and corn safrinha plots in August 2020. Signal grass is grown as a grazing crop, therefore no yield was taken from the signal grass plots in 2019.

Data were analyzed to assess whether P. brachyurus population densities and grain yield differed between plots following GMB151 soybean as compared to conventional soybean. Data were analyzed separately for each safrinha crop; corn, cotton, and signal grass. All data were analyzed using a mixed effects ANOVA model. Models included the fixed effects of location, GMB151 zygosity, and their interaction. Experimental replication was considered a random effect.

### DISCUSSION

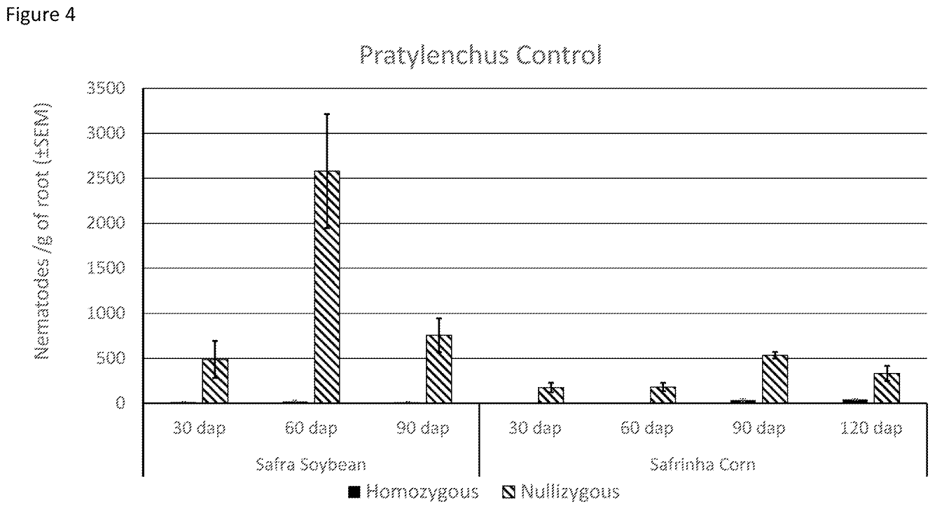

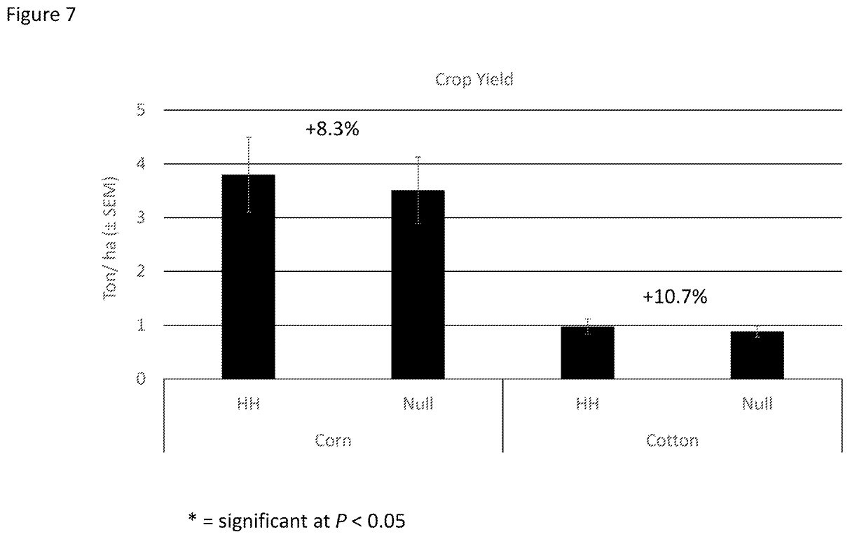

Pratylenchus brachyurus population densities were significantly reduced in all three safrinha crops by the prior planting of GMB151 soybean during the safra season. Depending on the safrinha crop, trial location, and sampling time point, P. brachyurus population densities were reduced by 28-100%, with an average reduction of 87%. There was no clear indication that P. brachyurus population suppression decreased over the course of the safrinha season in any of the three crops or that there was a resurgence of the nematode population. Therefore, planting GMB151 soybean reduced P. brachyurus populations throughout the entire cropping system (see e.g., FIG. 8), beginning with the safra soybean crop and extending to the safrinha crop harvest. GMB151 soybean reduced P. brachyurus populations by 99% at the end of the soybean season, which led to significant P. brachyurus suppression throughout the entire safrinha crop season, from planting through harvest of the corn safrinha crop (FIG. 4 and FIG. 7) and the cotton safrinha crop (FIG. 7).

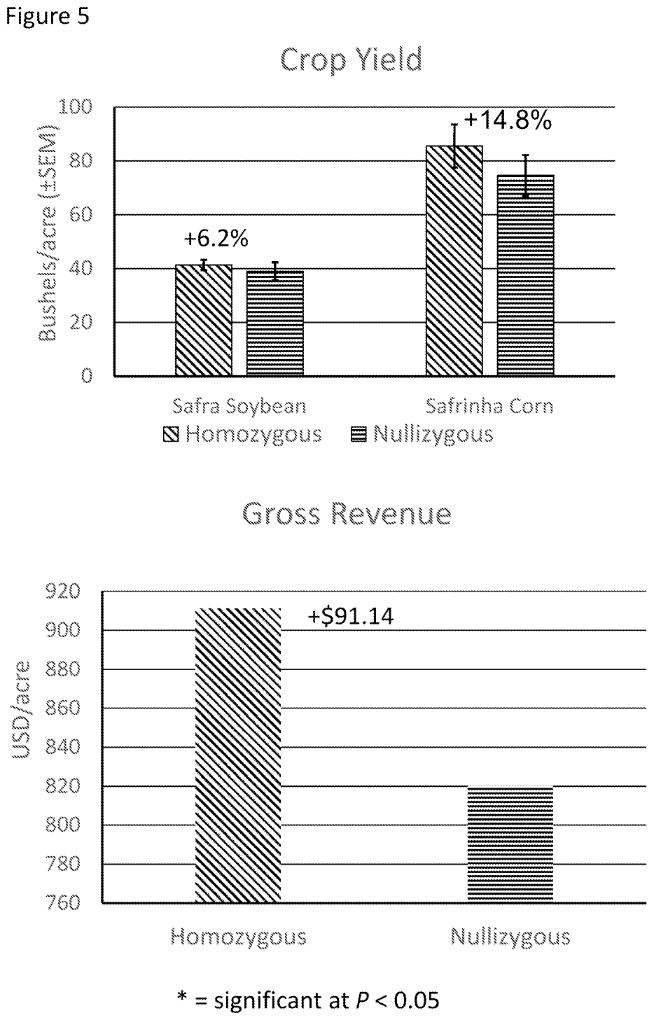

The safrinha crop protection resulted in substantial yield improvement in the case of corn production in Rio Verde, Mato Grosso. For example, as shown in FIG. 5, P. brachyurus control provided by GMB151 soybean resulted in numerically higher soybean yield, and significantly greater corn yield in the safrinha cropping season. The yield improvements provided by GMB151 from P. brachyurus control were worth approximately $90 USD/acre in increased gross revenue to the farmer. Gross revenue estimates are based on average Brazilian grain prices of $9.00 per bushel of soybean, and $6.30 per bushel of corn.

The data provided in this Example demonstrates that incorporating a high efficacy transgenic nematicidal protein into a host crop can provide protection to not only the transformed crop, but also to susceptible crops rotated with, or subsequently planted in the same locus as, the transformed crop. This provides an efficient method to provide nematode control across an entire farm production system. Transforming a single crop to provide protection to an entire production system limits the development and regulatory costs associated with producing and registering a transgenic crop. It also provides a cost-effective method to bring nematode resistance into crops where native resistance is not available, an especially common issue with migratory plant-parasitic nematodes such as Pratylenchus spp. This approach also provides an opportunity to bring transgenic nematode protection to crops, such as wheat, where end user acceptance of transgenic grain may be a barrier yet Pratylenchus spp. can be a significant yield-reducing pathogen with few effective management options available.

In one embodiment, transgenic GMB151 soybean brings significant yield and cropping system management benefits to Brazilian growers. The high efficacy of GMB151 against P. brachyurus provides growers with safra soybean protection and protection to multiple safrinha crop species. It is this benefit to the safrinha crop that also provides growers with freedom and flexibility in their production system to select the most profitable and agronomically advantageous rotation, regardless of crop susceptibility to P. brachyurus.

### REFERENCES FOR EXAMPLE 3

Barker, K. R., and S. R. Koenning. 1998. Developing sustainable systems for nematode management. Annual Review of Phytopathology 36: 165-205.
Cook, R. 2004. Genetic resistance to nematodes: where is it useful? Australasian Plant Pathology 33: 139-150.
Inagaki, H., and M. Tsutsumi. 1971. Survival of the soybean cyst nematode Heterodera glycines Ichinohe (Tylenchida: Heteroderidae) under certain storage conditions. Applied Entomology and Zoology 6: 156-162.
IRAC. 2018. Nematicide resistance risk statement. https://irac-online.org/teams/nematodes.
Nicol, J. M., S. J. Turner, D. L. Coyne, L. den Nijs, S. Hockland, and Z. T. Maafi. 2011. Current nematode threats to world agriculture, in: J. Jones, G. Gheysen, C. Fenoll (Eds.), Genomics and Molecular Genetics of Plant-Nematode Interaction, Springer, Germany, pp. 21-43.
Wei, J., K. Hale, L. Carta, E. Platzer, C. Wong, S. Fang, and F. V. Aroian. 2003. Bacillus thuringiensis crystal proteins that target nematodes. Proceedings of the National Academy of Sciences 100: 2760-2765.

### Example 4—Control of the Root Lesion Nematode Pratylenchus brachyurus (Godfrey) by GMB151 Transgenic Soybean Expressing the Bacillus thuringiensis Toxin Cry14Ab-1

Expanding on the experiments described in Example 2, the ability of Cry14Ab-1 to control P. brachyurus in the field was further investigated. Specifically, it was contemplated that the Cry14Ab-1-expressing event GMB151 could reduce P. brachyurus reproduction in field grown soybean and that this reduction in P. brachyurus reproduction would result in significant yield protection.

### Materials and Methods:

Field trials were conducted in the states of Bahia, Goiás, Mato Gross, Paraná, and Sao Paulo, Brazil, during the 2018/19, 2019/20, and 2020/21 soybean growing seasons. In total 49 soybean trials were planted across the three safra seasons.

The GMB151 event was introgressed into multiple soybean backgrounds ranging from MG VI to IX, each adapted for production in Brazil. Introgression was accomplished by utilizing the Brazilian adapted background as the recurrent parent. For each background, at the BC2:F2 or BC3:F2 generation plants homozygous and nullizygous for the GMB151 event were selected to create two soybean lines that differed in the presence or absence of the GMB151 event, but otherwise were approximately 87.5% BC2:F2) or 93.75% (BC3:F2) genetically related to the recurrent parent.

Soybean trials were established at research sites infested with a range of population densities of P. brachyurus.

Differences in experimental design and trial treatments differed among the trial sites. Each of the 49 trial sites included two main plot treatments replicated in a randomized complete block design with five to six replications. The two main plot treatments included in every trial were (1) a BC2:F2 or BC3:F2 line homozygous for the GMB151 event, and (2) the respective BC2:F2 or BC3:F2 line nullizygous for the GMB151 trait. Additional split-plot treatments were included at a subset of locations. Nine locations received split-plot treatments of chemical nematicide treatments. At these nine locations, each soybean line was treated with (1) a base fungicide/insecticide seed treatment, (2) a base fungicide/insecticide seed treatment and a nematicide seed treatment, and (3) a base fungicide/insecticide seed treatment and an in-furrow application of a liquid nematicide. Three other locations utilized a slightly different trial design in which the main plot treatment was level of tillage, either conventional tillage or no-till production. The split-plot treatment at these three sites was then soybean line, either homozygous for the GMB151 trait or nullizygous.

At all locations plots were 5 m in length and four rows wide.

### Data Collection:

The efficacy of GMB151, chemical nematicides, and tillage against P. brachyurus was evaluated by measuring the population density of P. brachyurus within soybean root systems at 90 days after planting (dap). The 90 dap time point was selected as it approximated the sampling time of previous P. brachyurus studies (Lima et al. 2015). At 90 dap the soybean root systems of ten plants per plot were removed from the field. Three to five root systems were sampled from the first and fourth row of each plot. These rows were selected as they were not harvested and therefore P. brachyurus sampling would not affect yield estimates taken at the end of the season. Root samples were taken to the laboratory where they were weighed and then homogenized in a blender. Nematodes were then extracted from the root pieces using the methods of Jenkins (1964). Extracted nematodes were then suspended in water and the number of P. brachyurus in a 1 ml subsample was enumerated under a microscope. The number of P. brachyurus per g of root was calculated for each sample.

Soybean yield was estimated for each plot at physiological maturity. The middle two rows of each four-row plot were harvested. Grain weight and moisture were measured for each plot and yield was standardized to 13% moisture.

### Data Analysis:

Pratylenchus brachyurus population density and yield data were analyzed first across all locations to estimate the effect of GMB151 on P. brachyurus population densities. The number of P. brachyurus per gram (g) of root was natural log transformed prior to analysis to reduce heteroscadacity. Yield data was analyzed as bushels per acre.

Pratylenchus brachyurus population density and yield data were then analyzed separately for the nine trials evaluating chemical nematicides in combination with GMB151. A third analysis was then completed to evaluate the three trial locations comparing tillage practices and the GMB151 trait for Pratylenchus brachyurus control and soybean yield protection.

### Results:

Pratylenchus brachyurus population densities on the GMB151 null line were above those observed to cause aboveground disease symptoms by Lima et al. (2015) in 33 of the 49 trials.

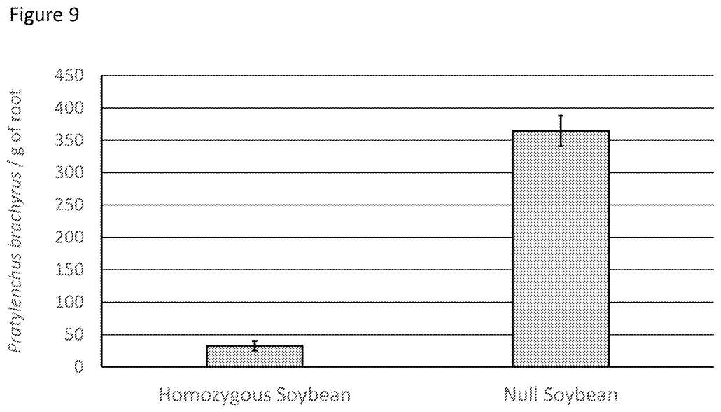

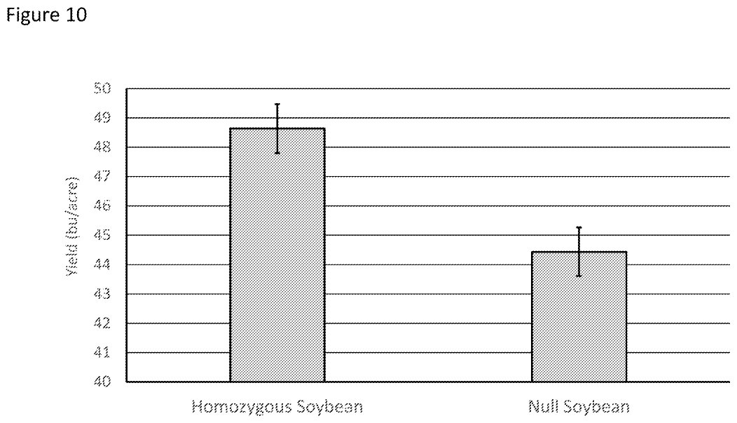

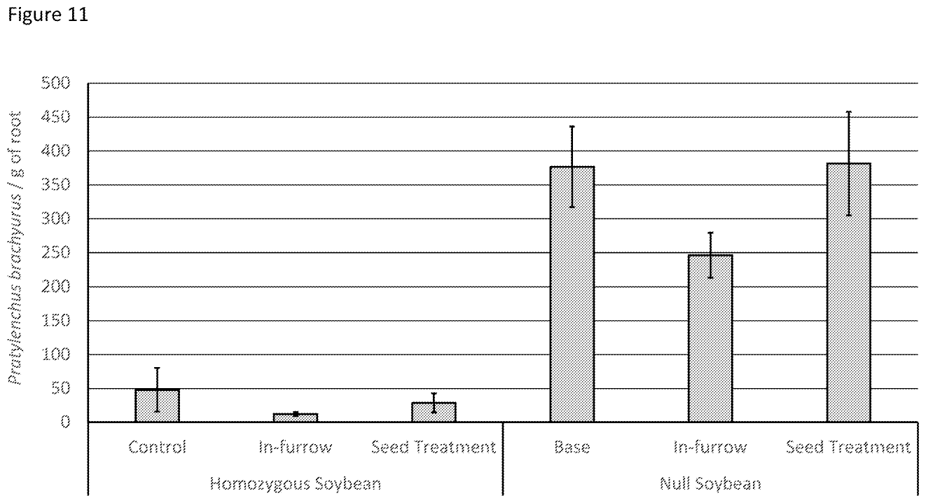

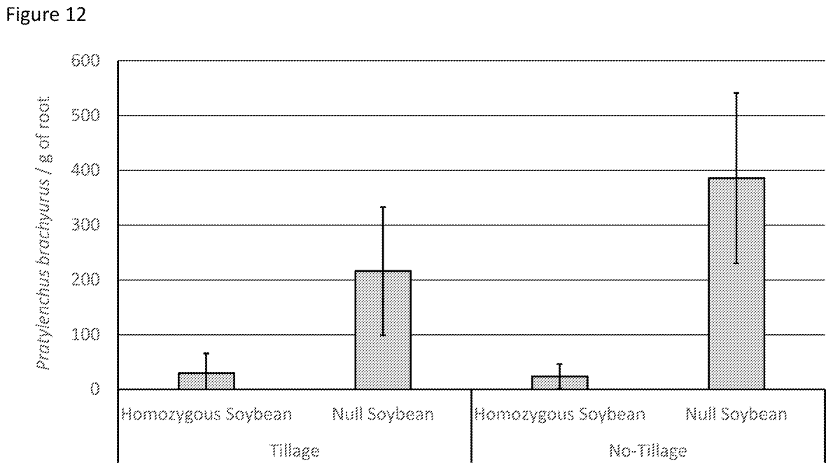

The Homozygous soybean line reduced P. brachyurus population densities by 91% across the 49 trials (P<0.0001) (FIG. 9). The GMB151 trait also significantly improved soybean yield by an average of 4.2 bu/acre or 9% (FIG. 10). The Homozygous line provided significantly better P. brachyurus control than either seed treatment or in-furrow nematicide applications (FIG. 11). The Homozygous line also provided significantly greater control than conventional tillage (FIG. 12).

The yield difference among Homozygous and Nullizygous lines was dependent upon the population density of P. brachyurus at the trial location with larger yield benefits generally observed in trials with higher P. brachyurus population densities. On average the Homozygous line had a soybean yield that was 4.2 bu/acre or 9.4% greater than the Null line (FIG. 2).

### DISCUSSION

The 49 research trials conducted ranged in Pratylenchus brachyurus population densities from near the limit of detection to nearly 1,000 nematodes per gram of root tissue. The trials allowed us to evaluate the efficacy of the GMB151 transgenic soybean event against P. brachyurus and its ability to protect soybean yield. The GMB151 event was highly efficacious against P. brachyurus. Three months into the growing season, P. brachyurus populations were >90% lower on average in the soybean line homozygous for the GMB151 event as compared to the nullizygous line. This level of control far exceeds that provided by current management practices, including crop rotation, fallowing, and chemical control (Lima et al. 2015, Ribeiro et al. 2014, Rodrigues et al. 2014).

The results of the field trials reported here indicate that the Cry14Ab1-expressing soybean trait GMB151 provided significant efficacy against P. brachyurus, resulting in improved soybean yields. These results were obtained under field production conditions with P. brachyurus population densities within the range reported for commercial production fields in Brazil (Franchini et al. 2007, Lima et al. 2015). Therefore, the GMB151 trait represents a potentially unique new management tool for controlling an economically important crop pest with few viable management options today.

### REFERENCES FOR EXAMPLE 4

Barbosa, B. F. F., J. M. dos Santos, J. C. Barbosa, P. L. M. Soares, A. R. Ruas, and R. B. de Carvalho. 2013. Aggressiveness of Pratylenchus brachyurus to the sugarcane, compared with key nematode P. zeae. Nematropica 43: 119-130.
Dias, W. P., N. R. Ribeiro, I. O. N. Lopes, A. Garcia, G. E. S. Carneiro, and J. F. V. Silva. 2007. Manejo de nematoides na cultura da soja. In: Congresso Brasileiro de Nematologia; 12 August; Goiâna, Brazil. Goiana: Sociedade Brasileira de Nematologia. p 26-30.
Franchini, J. C., O. F. Saraiva, H. Debiase, and S. L. Goncalves. 2007. Contribuição de sistema de manejo do solo para produção sustentável da soja. Circular Técnica 46:1-4.
Inomoto, M. M., A. M. C. Goulart, A. C. Z. Machado, and A. R. Monteiro. 2001. Effect of population densities of Pratylenchus brachyurus on the growth of cotton plants. Fitopatologia Brasileira 26: 192-196.
Jenkins, W. R. 1964. A rapid centrifugal-flotation technique for separating nematodes from soil. Plant Disease Reporter 48: 692.
Lima, F. S. D. O., G. R. D. Santos, S. R. Nogueira, P. R. R. D. Santos, and V. R. Correa. 2015. Population dynamics of the root lesion nematode, Pratylenchus brachyurus, in soybean fields in Tocantins State and its effect to soybean yield. Nematropica 45: 170-177.
Lima, F. S. O., V. R. Correa, S. R. Nogueira, and P. R. R. Santos. 2017. Nematodes affecting soybean and sustainable practices for their management. Intech doi: 10.5772/67030.
Ribeiro, N. R., W. P. Dias, and J. M. Santos. 2010. Distribuição de fitonematoides em regiões produtoras de soja no estado do Mato Grosso. Boletim de Pesquisa de Soja. Fundação MT, Rondonópolis, MT, 289-296.
Ribeiro, L. M, H. D. Campos, C. R. Dias-Arieira, D. L. Neves, and G. C. Ribeiro. 2014. Effect of soybean seed treatment on the population dynamics of Pratylenchus brachyurus under water stress conditions. Bioscience Journal 30: 616-622.
Rios, A. D. F., M. R. Rocha, A. S. Machado, K. A. G. B. Avila, R. A. Teixeira, L. C. Santos, and L. R. S. Rabelo. 2016. Host suitability of soybean and corn genotypes to the lesion caused by nematode under natural infestation conditions. Ciência Rural. 46: 580-584.
Rodrigues, D. B., C. R. Dias-Arieira, M. V. V. Vedoveto, M. Roldi, H. F. D. Molin, and V. H. F. Abe. 2014. Crop rotation for Pratylenchus brachyurus control in soybean. Nematropica 44: 146-151.
Wei, J., K. Hale, L. Carta, E. Platzer, C. Wong, S. Fang, and F. V. Aroian. 2003. Bacillus thuringiensis crystal proteins that target nematodes. Proceedings of the National Academy of Sciences 100: 2760-2765.

### Example 5-GMB151 Transgenic Soybean Expressing the Bacillus thuringiensis Toxin Cry14Ab-1 Provides Nematicidal Protection in Safrinha (“Second Harvest”) Crops

Building upon and expanding the experiments described in Example 3, this Example sought to further determine whether planting GMB151 soybean in the safra season could provide nematode suppression to subsequent nematode susceptible safrinha season crops.

Two soybean lines, one homozygous and one null for the GMB151 trait, were planted in production fields during the safra production season in fields infested with P. brachyurus. Immediately after soybean trial harvest in April/May 2019, 2020, and 2021a conventional safrinha crop was planted on top of the same ground. Each safrinha crop trial consisted of three replicates arranged in a randomized complete block design. In 2019, Brachiaria (signal grass) was planted as the safrinha crop. Signal grass plots were planted parallel to harvested soybean rows at each location. Plots were 5 meters in length by 12 rows wide. In April 2020 and 2021, corn and cotton were planted as the safrinha crops following soybean. Three replications of corn plots and three replications of cotton plots were planted on top of the soybean trial ground. Corn and cotton rows were planted perpendicular to the harvested soybean rows. Cotton plots were planted on top of replications one, two, and the first three m of replication three. Corn plots were planted on top of replications five, four and the last three m of replication three. Cotton and corn plots were therefore 9 m in length, with two replications being 8 rows wide, and the third replication of each crop being 4 rows wide (FIG. 3). In 2021, at three location the cotton safrinha trial was replaced with a safrinha trial of sorghum.

Data were collected to determine if protection from P. brachyurus would be provided to the susceptible safrinha crops planted following the GMB151 soybean. Protection to safrinha crops was assessed by measuring P. brachyurus population densities and crop yield. Pratylenchus brachyurus population densities were measured as the number of nematodes per g of root tissue. Population densities of P. brachyurus were measured at 30 days after planting (dap), 60 dap and 90 dap in the safrinha crops. The root system of ten plants per plot were collected for each P. brachyurus measurement. Three to five roots were collected from the first row and last row of each plot. The middle rows were not sampled as root samples are destructive and would interfere with the collection of yield data from the middle rows of each plot. Grain yield was collected from the middle rows of the cotton, corn, and sorghum safrinha plots in August 2020 and 2021. Brachiaria is grown as a grazing crop, therefore no yield was taken from the signal grass plots in 2019.

Data were analyzed to assess whether P. brachyurus population densities and grain yield differed between plots following GMB151 soybean as compared to conventional soybean. Data were analyzed separately for each safrinha crop; corn, cotton, sorghum and brachiaria. All data were analyzed using a mixed effects ANOVA model. Models included the fixed effects of location, GMB151 zygosity, and their interaction. Experimental replication was considered a random effect.

### DISCUSSION

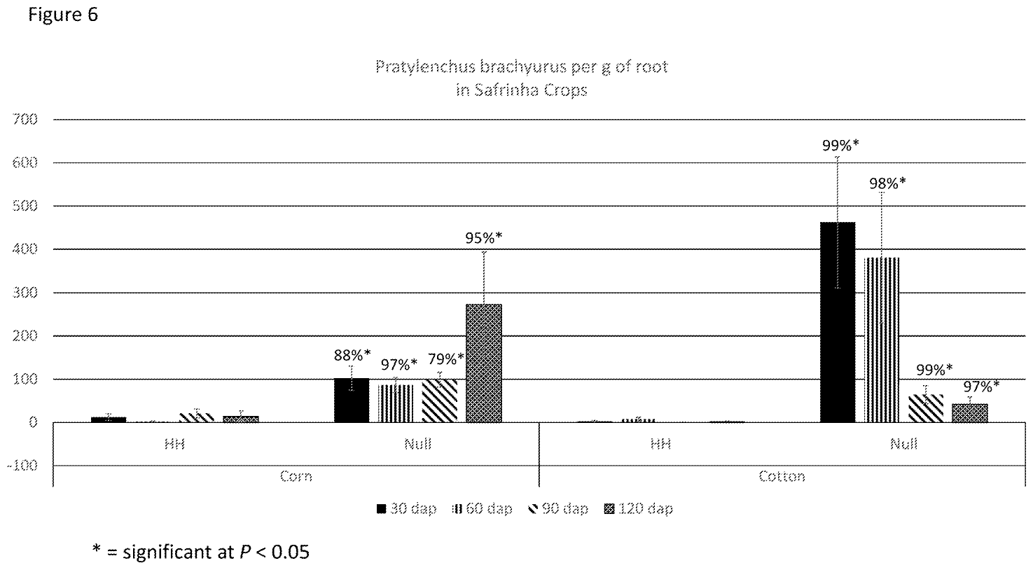

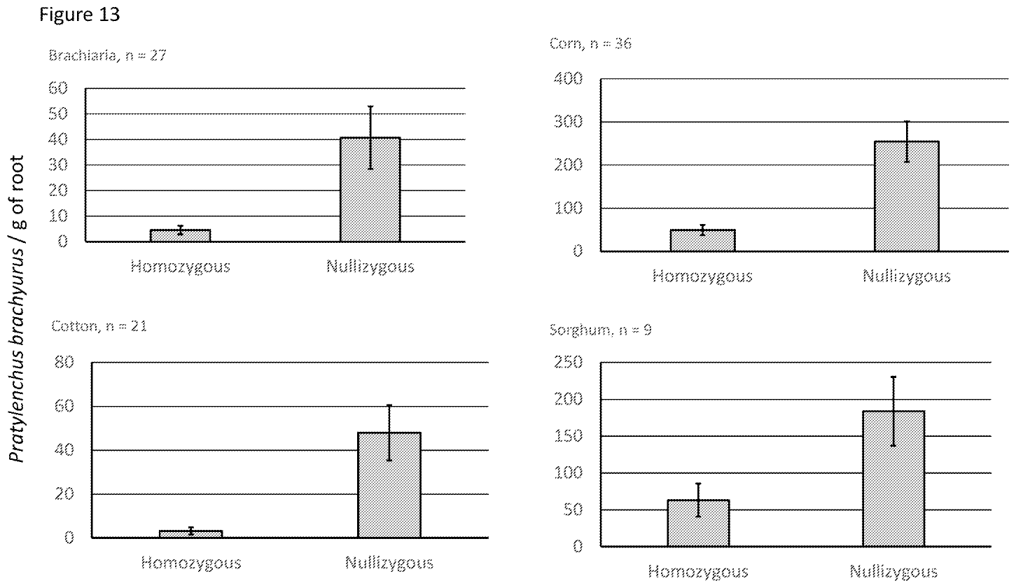

Pratylenchus brachyurus population densities were significantly reduced in all four safrinha crops by the prior planting of GMB151 soybean during the safra season (FIG. 13). Depending on the safrinha crop, brachyurus population densities were reduced by an average of 66-93% 90 days after planting, with an average reduction of 80%. There was no clear indication that P. brachyurus population suppression decreased over the course of the safrinha season in any of the three crops or that there was a resurgence of the nematode population. Therefore, planting GMB151 soybean reduced P. brachyurus populations throughout the entire cropping system (see e.g., FIG. 8), beginning with the safra soybean crop and extending to the safrinha crop harvest. This lead to improvements in the yield of both the safra soybean crop (FIG. 10) and the subsequent safrinha crops (FIG. 7). At this example location GMB151 soybean reduced P. brachyurus populations by 99% at the end of the soybean season, which led to significant P. brachyurus suppression throughout the entire safrinha crop season, from planting through harvest of the corn safrinha crop (FIG. 4 and FIG. 6) and the cotton safrinha crop (FIG. 6).

The safrinha crop protection resulted in substantial yield improvement in the case of corn production in Rio Verde, Mato Grosso. For example, as shown in FIG. 5, P. brachyurus control provided by GMB151 soybean resulted in numerically higher soybean yield, and significantly greater corn yield in the safrinha cropping season. The yield improvements provided by GMB151 from P. brachyurus control were worth approximately $90 USD/acre in increased gross revenue to the farmer. Gross revenue estimates are based on average Brazilian grain prices of $9.00 per bushel of soybean, and $6.30 per bushel of corn.

The data provided in Example 3 and in this Example 5 demonstrate that incorporating a high efficacy transgenic nematicidal protein into a host crop can provide protection to not only the transformed crop, but also to susceptible crops rotated with, or subsequently planted in the same locus as, the transformed crop. This provides an efficient method to provide nematode control across an entire farm production system. Transforming a single crop to provide protection to an entire production system limits the development and regulatory costs associated with producing and registering a transgenic crop. It also provides a cost-effective method to bring nematode resistance into crops where native resistance is not available, an especially common issue with migratory plant-parasitic nematodes such as Pratylenchus spp. This approach also provides an opportunity to bring transgenic nematode protection to crops, such as wheat, where end user acceptance of transgenic grain may be a barrier yet Pratylenchus spp. can be a significant yield-reducing pathogen with few effective management options available.

In one embodiment, transgenic GMB151 soybean brings significant yield and cropping system management benefits to Brazilian growers. The high efficacy of GMB151 against P. brachyurus provides growers with safra soybean protection and protection to multiple safrinha crop species. It is this benefit to the safrinha crop that also provides growers with freedom and flexibility in their production system to select the most profitable and agronomically advantageous rotation, regardless of crop susceptibility to P. brachyurus.

Effects shown herein with respect to one nematode-susceptible plant would also be expected on hosts having the same susceptibility to nematodes. Below is a table showing the susceptibility of some common crops:

| | Brazilian Acreage | P. Brachyurus |
|---|---|---|
| Crop | (million acres) | Host Suitability |
| Signal Grass^{‡} | 251.9 | Good host |
| Soybean | 99.8 | Good host |
| Corn | 51.4 | Good host |
| Millet* | 12.4 | Weak host |
| Common Beans | 7.2 | Good host |
| Wheat | 6.7 | Good host |
| Rice | 4.2 | Good host |
| Cotton | 4.0 | Good host |
| Sorghum | 2.2 | Good host |
| Oat | 1.1 | Good host |
| Sunflower | 0.1 | Weak host |

^{‡}Estimate of grazing land acreage
*Includes cover crop acres

For cropping acres estimates, see also, Camila Thaiana Rueda da Silva et al., Agriculture 2020, 10, 13; doi:10.3390/agriculture10010013; Renato Lara De Assis et al., Expl Agric.: page 1 of 20 ©Cambridge University Press 2017 doi:10.1017/S0014479717000333; and U.S. Department of Agriculture, “World Agricultural Production”, Foreign Agriculture Service, Global Market Analysis, Circular Series WAP 2-22, February 2022.

### References for Example 5

Barker, K. R., and S. R. Koenning. 1998. Developing sustainable systems for nematode management. Annual Review of Phytopathology 36: 165-205.
Cook, R. 2004. Genetic resistance to nematodes: where is it useful? Australasian Plant Pathology 33: 139-150.
Inagaki, H., and M. Tsutsumi. 1971. Survival of the soybean cyst nematode Heterodera glycines Ichinohe (Tylenchida: Heteroderidae) under certain storage conditions. Applied Entomology and Zoology 6: 156-162.
IRAC. 2018. Nematicide resistance risk statement. https://irac-online.org/teams/nematodes.
Nicol, J. M., S. J. Turner, D. L. Coyne, L. den Nijs, S. Hockland, and Z. T. Maafi. 2011. Current nematode threats to world agriculture, in: J. Jones, G. Gheysen, C. Fenoll (Eds.), Genomics and Molecular Genetics of Plant-Nematode Interaction, Springer, Germany, pp. 21-43.
Wei, J., K. Hale, L. Carta, E. Platzer, C. Wong, S. Fang, and F. V. Aroian. 2003. Bacillus thuringiensis crystal proteins that target nematodes. Proceedings of the National Academy of Sciences 100: 2760-2765.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

1. A method of suppressing a nematode population in a locus, the method comprising:
growing a nematode-resistant plant in a locus, wherein growing the nematode-resistant plant suppresses a nematode population in the locus or maintains the suppression of the nematode population in the locus for a period of time during and/or beyond the growing of the nematode-resistant plant, wherein the period of time extends to one or more growing seasons subsequent to the growing season in which the nematode-resistant plant was grown.

2. (canceled)

3. The method of claim 1, further comprising growing a secondary plant in the locus subsequent to growing the nematode-resistant plant, or prior to growing the nematode-resistant plant in the locus, or growing the nematode-resistant plant at the same time as a secondary plant.

4. (canceled)

5. (canceled)

6. The method of claim 3, wherein the secondary plant is a nematode-susceptible plant.

7. The method of claim 1, wherein the nematode population is suppressed or maintained at or below the limit of detection.

8. The method of claim 3, wherein the secondary plant is Brachiaria and the suppressing of the nematode population is achieved when the number of nematodes is, is about or is less than about 60 nematodes per gram of root.

9. The method of claim 3, wherein the secondary plant is corn and the suppressing of the nematode population is achieved when the number of nematodes is, is about or is less than about 300 nematodes per gram of root.

10. The method of claim 3, wherein the secondary plant is cotton and the suppressing of the nematode population is achieved when the number of nematodes is, is about, or is less than about 60 nematodes per gram of root.

11. The method of claim 3, wherein the secondary plant is sorghum and the suppressing of the nematode population is achieved when the number of nematodes is, is about, or is less than about 250 nematodes per gram of root.

12. The method of claim 3, wherein the suppressing of the nematode population in the locus in which the secondary plant is grown is achieved when there is, is about, or is at least about a 5% reduction in the number of nematodes per gram of root relative to the number of nematodes per gram of root in a comparable locus to the locus in which the secondary plant is grown.

13. A method of protecting a nematode-susceptible plant from nematode injury or damage comprising:
growing a nematode-resistant plant in a locus at least one growing season before planting the nematode-susceptible plant; and
growing a nematode-susceptible plant in the locus at least one growing season subsequent to growing the nematode-resistant plant.

14. A method of increasing yield of a nematode-susceptible plant comprising:
growing a nematode-resistant plant in a locus at least one growing season before planting the nematode-susceptible plant; and
growing a nematode-susceptible plant in the locus at least one growing season subsequent to growing the nematode-resistant plant.

15. The method of claim 14, wherein the increased yield of the nematode-susceptible plant is compared to the yield of the nematode-susceptible plant grown in the same or a comparable locus in which the nematode-resistant crop has not been grown during the most recent crop rotation cycle.

16. The method of claim 3, wherein the nematode-susceptible plant is a perennial plant or an annual plant.

17. The method of claim 3, wherein the nematode-susceptible plant is a soybean plant, a corn plant, a cotton plant, a canola plant, a sugarcane plant, a potato plant, a wheat plant, a vegetable plant, a rice plant, an alfalfa plant, a barley plant, a sorghum plant, an oat plant, a rye plant, a cassava plant, a sweet potato plant, a sunflower plant, a fruit plant, an orchard plant (such as a fruit or nut tree), an ornamental plant, or a grapevine.

18. The method of claim 1, wherein the nematode-resistant plant expresses a nematicidal Cry protein.

19. The method of claim 1, wherein the nematode-resistant plant is a soybean plant, a corn plant, a cotton plant, a canola plant, a sugarcane plant, a sugar beet plant, a potato plant, a wheat plant, a rice plant, an alfalfa plant, a barley plant, a sorghum plant, an oat plant, a rye plant, a cassava plant, a sweet potato plant, a sunflower plant, a vegetable plant, a fruit plant, an ornamental plant, an orchard plant (such as a fruit or nut tree), or a grapevine.

20. The method of claim 1, wherein the nematode is a nematode species selected from Pratylenchus spp. nematode populations, e.g., Pratylenchus brachyurus, Meloidogyne spp., Heterodera spp., e.g. Heterodera glycines, Globodera spp., Rotylenchulus reniformis, Helicotylenchus spp., e.g. Helicotylenchus dihystera, Scutellonema brachyurus, Tubixaba tuxaua, or Aphelencoides besseyi.

21. (canceled)

22. A locus having a suppressed nematode population density, wherein the suppressed nematode population density is achieved by the method of claim 1.

23. (canceled)

24. The locus of claim 22, wherein the suppressed nematode population density is, is about, or is less than about 250, 200, 150, 100, 50, 20, or 10 nematodes per gram (g) of root.

25. A nematode-resistant plant grown in the locus of claim 22.

26. Plant material harvested from the plant of claim 25.

27. Seeds produced by the plant of claim 25.

28. A system for increased use of a locus, the system comprising:
growing a nematode-resistant plant in a locus in a first growing season; and
growing a nematode-susceptible plant in the locus in a subsequent growing season.

29. The system of claim 28, wherein the locus is not fallowed during a subsequent growing season.

30. The system of claim 28, wherein no cover plant or cover crop is grown during the subsequent growing season.

31. The system of claim 28, wherein the nematode-resistant plant and the nematode-susceptible plant are grown in consecutive growing seasons.

32. The system of claim 28, wherein each of the nematode-resistant plant and the nematode-susceptible plant is an intrinsic value crop plant.

33. A method for improving a crop rotation system, the method comprising:
growing a nematode-resistant plant in a locus during a first growing season; and
growing a nematode-susceptible plant in the locus during a subsequent growing season, wherein growing the nematode-resistant plant in the locus during the first growing season results in a suppression of the nematode population in the locus that enables or improves the growth of the nematode-susceptible plant in the subsequent growing season.

34. The method of claim 33, wherein the improved crop rotation system may further include one or more of the following:
a. use of the locus for at least one additional growing season per year;
b. decreased tillage of the locus;
c. decreased treatment of the nematode-susceptible crop seed with nematicides;
d. decreased treatment area of the locus with nematicides prior to or during the growing season for the nematode-susceptible plant;
e. decreased rate of nematicide applied to the nematode-susceptible plant and/or the locus prior to or during the growing season for the nematode-susceptible plant;
f. decreased number of applications of nematicides made to the nematode-susceptible plant and/or the locus during the growing season;
g. increased usability of the locus;
h. increased value of the locus;
i. improved sustainable agricultural practices; and/or
j. increased yield from the nematode-susceptible crop.

35. A method of nematode management for a locus, the method comprising:
growing a nematode-resistant plant in a locus in a first growing season, wherein growing the nematode-resistant plant in the first growing season suppresses a nematode population in the locus or maintains the suppression of the nematode population in the locus;
growing a nematode-susceptible plant in the locus in the same or a subsequent growing season; and
achieving improved health and/or yield of the nematode-susceptible plant compared to the health and/or yield expected if the nematode population was not suppressed.

36. The method of claim 35, wherein the improved health of the nematode-susceptible plant comprises one or more of the following: improved root development (e.g., improved root or root hair growth); improved yield; faster emergence; improved plant stress management including increased stress tolerance and/or improved recovery from stress; increased mechanical strength; improved drought resistance; reduced fungal, bacterial and/or viral disease infection; or any combination thereof.

37. A method for marketing a crop rotation system, the method comprising:
promoting use of a nematode-resistant plant during a first growing season; and
promoting use of a nematode-susceptible plant in the locus during the same or a subsequent growing season, wherein growing the nematode-resistant plant in the locus during the first growing season results in a suppression of the nematode population in the locus that enables or improves the growth of the nematode-susceptible plant in the subsequent growing season.

38. The method of claim 37, wherein the subsequent growing season is immediately adjacent to the first growing season.

39. Marketing materials directed to a crop rotation system of growing a nematode-resistant plant during a first growing season in coordination with or followed by growing a nematode-susceptible plant in the locus during a subsequent growing season, wherein growing the nematode-resistant plant in the locus during the first growing season results in a suppression of the nematode population in the locus that enables or improves the growth of the nematode-susceptible plant.

40. The marketing materials of claim 39, wherein the marketing materials are directed to promoting the crop rotation system.

41. A method of nematode management for a locus, the method comprising: growing a nematode-resistant plant in a locus prior to, at the same time as, or after growing a secondary crop in the locus.

42. The method of claim 1, wherein the nematode-resistant plant expresses a nematicidal Cry14Ab protein having at least 95, 96, 97, 98, or at least 99% sequence identity to SEQ ID NO. 1.

43. The method of claim 1, wherein the nematode-resistant plant comprises elite event EE-GM5.

44. The method of claim 1, wherein the nematode-resistant plant comprises elite event EE-GM4.

45. (canceled)

46. (canceled)

47. (canceled)
